(19) United States
(12) Patent Application Publication
(10) Pub. No.: US 2023/0138742 A1
(43) Pub. Date: May 4, 2023

(54) STERILE BARRIER FOR MEDICAL IMAGING DEVICE

(71) Applicant: Lumicell, Inc., Newton, MA (US)

(72) Inventors: Dan Harris, Newton Highlands, MA (US); Jorge Ferrer, West Newton, MA (US); David Benjamin Strasfeld, Somerville, MA (US); Matthias Hofmann, Wayland, MA (US); Samantha Higer-Grunes, Needham Heights, MA (US)

(73) Assignee: Lumicell, Inc., Newton, MA (US)

(21) Appl. No.: 17/977,215

(22) Filed: Oct. 31, 2022

Related U.S. Application Data
(60) Provisional application No. 63/275,869, filed on Nov. 4, 2021.

(51) Int. Cl. A61B 46/17; A61B 5/00

(52) U.S. Cl. CPC A61B 46/17; A61B 5/0071; A61B 2505/05

(57) ABSTRACT

Embodiments related to medical imaging devices including rigid imaging tips and their methods of use for identifying abnormal tissue within a surgical bed are disclosed. A barrier for an imaging device may include an optically transparent window configured to be removably attached to a distal portion of the imaging device. The barrier may also include a flexible barrier secured to the optically transparent window configured to cover portions of the imaging device located proximal relative to the optically transparent window. The barrier may include a second flexible barrier secured to the optically transparent window and disposed over at least a portion of the first flexible barrier.

### RELATED APPLICATIONS

This application claims the benefit of priority under 35 U.S.C. § 119(e) of U.S. Provisional Application Ser. No. 63/275,869, filed Nov. 4, 2021, the disclosure of which is incorporated herein by reference in its entirety.

### FIELD

Disclosed embodiments are related to sterile barriers for medical imaging devices and related methods of use.

### BACKGROUND

There are over one million cancer surgeries per year performed in the United States and nearly 40% of them miss resecting the entire tumor according to the National Cancer Institute Surveillance Epidemiology and End Results report. For example, in breast cancer lumpectomies, failure to remove all of the cancer cells during the primary surgery (positive margins) occurs approximately 50% of the time and requires second surgeries. Residual cancer in the surgical bed is a leading risk factor for local tumor recurrence, reduced survival rates and increased likelihood of metastases. In addition, final histopathology of the resected tumor misses 25% of the residual cancer left in the surgical bed, which must be addressed with adjuvant medical therapy (e.g., radiotherapy or chemotherapy). This poor performance of pathology is primarily due to a sampling error since only a small fraction of the entire resection is analyzed.

In a typical solid tumor resection, the surgeon removes the bulk of the tumor and sends it to pathology. The pathologist then samples the bulk tumor in a few locations and images a stained section under a microscope to determine if the surgeon has completely removed all of cancer cells from the patient. Should the pathologist find a portion of the stained sample with cancer cells bordering ink (a diagnostic known in the medical realm as “positive margin”), the surgeon may be instructed to resect more tissue. However, this pathology exercise is a time intensive procedure and often takes days for final results to be sent to the physician. Should a pathology report requiring additional resection return after the patient has completed the initial surgery, this may require the surgeon to perform a second surgery.

### SUMMARY

In some embodiments, a barrier for an imaging device includes an optically transparent window configured to be removably attached to a distal portion of the imaging device, and a flexible barrier secured to the optically transparent window configured to cover portions of the imaging device located proximal relative to the optically transparent window.

In some embodiments, a barrier for an imaging device includes a cap configured to be removably attached to a distal portion of the imaging device, a first flexible barrier secured to the cap configured to cover portions of the imaging device located proximal relative to the cap, and a second flexible barrier secured to the cap disposed over at least a portion of the first flexible barrier.

In some embodiments, a barrier for an imaging device includes an optically transparent window including one or more connectors, where the optically transparent window is configured to be removably attached to a distal portion of the imaging device, and a housing containing the optically transparent window, where the housing includes at least one resilient protrusion configured to position the optically transparent window in the housing to receive the distal portion of the imaging device.

In some embodiments, a method of installing a barrier on an imaging device includes removably attaching an optically transparent window to a distal portion of the imaging device, and covering portions of the imaging device located proximal relative to the optically transparent window with a flexible barrier secured to the optically transparent window.

In some embodiments, a method of installing a barrier for an imaging device includes removably attaching a cap to a distal portion of the imaging device, covering portions of the imaging device located proximal relative to the cap with a first flexible barrier secured to the cap, and covering at least a portion of the first flexible barrier with a second flexible barrier secured to the cap.

In some embodiments, a method of installing a barrier for an imaging device includes obtaining an optically transparent window within a housing with at least one resilient protrusion, where the at least one resilient protrusion positions the optically transparent window within the housing, and inserting a distal portion of the imaging device into the housing to removably attach the optically transparent window to the distal portion of the imaging device. The method also includes deflecting the at least one resilient protrusion to release the optically transparent window from the housing.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of a surgical bed being imaged with decreased magnification;

FIG. 2 is a perspective view of one embodiment of a handheld medical imaging device;

FIG. 3 is a partially exploded view of one embodiment of a probe of a handheld medical imaging device;

FIG. 4A is a side cross-sectional view taken along line 4A-4A of FIG. 3;

FIG. 4B is a perspective cross-sectional view taken along line 4A-4A of FIG. 3;

FIG. 5A is a perspective view of one embodiment of a sterile barrier package;

FIG. 5B is a perspective view of the sterile barrier package of FIG. 5A with a lid removed;

FIG. 6 is a partially exploded view of one embodiment of a sterile barrier assembly;

FIG. 7 is a cross-sectional view of the sterile barrier assembly of FIG. 6 taken along lines X-X;

FIG. 8 is a perspective view of one embodiment of a removable cap;

FIG. 9 is a side view of the removable cap of FIG. 8 and one embodiment of a flexible barrier;

FIG. 10 is a cross-sectional view of the removable cap of FIG. 8 taken along line Y-Y;

FIG. 11A depicts one embodiment of a medical imaging device and a sterile barrier assembly in a first state;

FIG. 11B depicts the medical imaging device and sterile barrier assembly of FIG. 11A in a second state;

FIG. 11C depicts the medical imaging device and sterile barrier assembly of FIG. 11A in a third state;

FIG. 11D depicts the medical imaging device and sterile barrier assembly of FIG. 11A in a fourth state;

FIG. 12A is a schematic of another embodiment of a sterile barrier assembly in a first state;

FIG. 12B is a schematic of the sterile barrier assembly of FIG. 12A in a second state;

FIG. 13 is a flow chart for one embodiment of a method of installing a sterile barrier on a medical imaging device; and

FIG. 14 is a flow chart for one embodiment of a method of assembling a sterile barrier.

### DETAILED DESCRIPTION

According to exemplary embodiments described herein, a medical imaging device may be employed to detect the presence of abnormal tissue during a surgical procedure, such as removal of cancerous cells or other aberrational cells. The medical imaging device may be reusable with multiple patients across multiple surgical procedures. In some instances, a medical imaging device may be in close proximity to or contact with a surgical site, such that the sterility of the medical imaging device is desirable or, in some cases, necessary. The inventors have appreciated that conventional sterilization methods employ high temperatures (e.g., steam), gases, or acids which may degrade or damage sensitive optics of the medical imaging device. Moreover, the inventors have appreciated that even if the medical imaging device is sterilized between uses using conventional methods, it may still be desirable to provide an additional sterile barrier between the patient and the device. Such an additional sterile barrier may be particularly desirable in instances where the device contacts tissue and foreign material may be deposited on the imaging device. The inventors have also appreciated the desirability of a sterile barrier that does not interfere with the quality or accuracy of the medical imaging device.

In view of the above, the inventors have appreciated the benefits of a sterile barrier assembly for a medical imaging device. In particular, the inventors have appreciated the benefits of a sterile barrier assembly that may be simple for a user to deploy and use with a medical imaging device. The inventors have also appreciated the benefits of a sterile barrier that does not interfere with the image or detection quality of the medical imaging device. For example, the sterile barrier may be optically transparent in the wavelengths of interest and may ensure the target tissue is still within a depth of field of the imaging device. In some embodiments, the sterile barrier may be removably attached to the imaging device, such that the sterile barrier may be attached prior to use of the imaging device and removed after use of the imaging device. Several embodiments of sterile barriers are discussed herein.

In some embodiments, a barrier for an imaging device includes an optically transparent window configured to be removably attached to a distal portion (e.g., a distal end portion) of the imaging device. For example, in some embodiments, the window may include one or more resilient latches configured to engage the distal portion of the imaging device (e.g., in snap-fit arrangement). As another example, in some embodiments, the window may include a magnet configured to magnetically attach to the distal portion of the imaging device. Of course, any suitable connection may be employed between a window and a distal portion of an imaging device, including, but not limited to, snap on connection, screw on connection, suction connection, magnetic connection, adhesive connection, and/or any other appropriate type of connection. In some embodiments, the barrier for an imaging device may also include a flexible barrier secured to the optically transparent window that is configured to cover portions of the imaging device located proximal relative to the optically transparent window.

In some embodiments, the optically transparent window of a barrier may be an independent component. For example, the optically transparent window may be directly attachable to a distal portion of an imaging device. Additionally, a flexible barrier may be attached to (e.g., directly attached to) to the optically transparent window. In some embodiments, the optically transparent window may be included as a part of a cap. For example, in some embodiments, the optically transparent window may be disposed in the cap. In some embodiments, the cap may be annular, and the optically transparent window may be disposed in an opening of the cap. In some such embodiments, the optically transparent window may close an end of the cap. In some embodiments, a cap may include one or more integrated connectors (e.g., resilient latches, magnets, etc.) configured to releasably attach the optically transparent window to a distal portion of an imaging device. According to such embodiments, the optically transparent window may be secured to the distal portion of the imaging device indirectly through the cap. In some embodiments, a flexible barrier may be attached to (e.g., directly attached to) to the cap. According to exemplary embodiments described herein, an optically transparent window may be employed with or without a corresponding cap. Additionally, according to exemplary embodiments described herein, a cap may be employed with or without an optically transparent window. Embodiments described herein described in reference to an optically transparent window may include a corresponding cap or not, as the present disclosure is not so limited.

In some embodiments, a flexible barrier secured to an optically transparent window and/or cap may be a thin flexible film (e.g., a plastic film). The flexible barrier may be secured to the optically transparent window and/or cap via any appropriate connection. For example, in some embodiments the flexible barrier may be secured to the optically transparent window and/or cap via ultrasonic welding. As another example, in some embodiments the flexible barrier may be secured to the optically transparent window and/or cap with an adhesive. As still another example, in some embodiment the flexible barrier may be secured to the optically transparent window and/or cap via a press fit (e.g., interference fit) between the window and/or cap and a press fit component such as a press fit ring. Of course, any suitable arrangement to secure the flexible barrier to the window and/or cap may be employed, including, but not limited to, adhesives, heat welding, fasteners (e.g., screws, rivets, tacks, etc.), press fit, ultrasonic welding, chemical welding, and/or any other appropriate type of connection. In some embodiments, the connection between the window and/or cap and the flexible barrier may be permanent, such that the flexible barrier is not removable from the window and/or cap without at least partially destroying the flexible barrier. In some embodiments, the flexible barrier may be secured to a periphery (e.g., a perimeter) of the optically transparent window, such that an interior portion of the window may remain uncovered. According to such embodiments, the flexible barrier may not be disposed in an optical path of the imaging device. Of course, in some embodiments the flexible barrier may cover at least a portion of the window, as the present disclosure is not so limited.

In some embodiments, a barrier for an imaging device may include a housing. The housing may be configured to contain an optically transparent window and at least one flexible barrier. In some embodiments, the housing may facilitate deployment of the optically transparent window so that the window may be releasably attached to a distal portion of the imaging device. In some embodiments, the housing includes at least one resilient protrusion configured to engage the optically transparent window to position the window in the housing. For example, in some embodiments, the at least one resilient protrusion may center the optically transparent window in the housing, for instance, along a central axis of the housing. In some embodiments, the central axis may be aligned with (e.g., parallel to) an insertion path of an imaging device through the housing. Of course, the at least one resilient protrusion may position the optically transparent housing in any suitable position, as the present disclosure is not so limited. In some embodiments, the at least one resilient protrusion may be configured to release the optically transparent window when a threshold force is applied to the optically transparent window. In some cases, such a threshold force may be applied with the imaging device. In some embodiments, the at least one resilient protrusion may be configured to deform (e.g., elastically), to allow the imaging device to pass through the housing. In some embodiments, a first end of the at least one flexible barrier may be secured to the optically transparent window, and a second end of the at least one flexible barrier may be secured to the housing. According to some such embodiments, the housing may be used to deploy the at least one flexible barrier on the imaging device. For example, in some embodiments, the housing may pass over the imaging device (e.g., the housing may receive the imaging device) so that the at least one flexible barrier may cover the imaging device at locations proximal to the optically transparent window as the housing is passed over the imaging device and any proximally located components such as cables extending out from the imaging device. In some embodiments, the housing may include a plurality of resilient protrusions. In some embodiments, the plurality of resilient protrusions may surround a periphery of the optically transparent window. For example, the plurality of resilient protrusions may apply force to the optically transparent window in multiple opposing directions (e.g., lateral direction) around the periphery of the window to position the optically transparent window in a desired location and orientation within the housing. In some embodiments, the at least one resilient protrusion may resist lateral movement of the optically transparent window. In some embodiments, the at least one resilient protrusion may be a pin, fin, or another suitable structure. Of course, the at least one resilient protrusion may have any suitable shape, as the present disclosure is not so limited.

In addition to the above, the inventors have appreciated that in some cases, it may be desirable to ensure tissue that is being imaged by an imaging device is disposed within a depth of field of the imaging device. The inventors have further appreciated that the position of the tissue in the depth of field may be ensured by placing a rigid component of the imaging device in contact with the tissue to be imaged, such that the distance between the tissue and a photosensitive detector of the imaging device is known. Accordingly, in some embodiments, an optically transparent window of a barrier may have a distally oriented surface. When connected to an imaging device, this distally oriented surface may be disposed within a predetermined distance of a focal plane of the photosensitive detector. This predetermined distance may be a depth of field of the imaging device when the window is removably attached to a distal portion of the imaging device. The optically transparent window may be configured to be placed in contact with tissue. Thus, when the distally oriented surface of the optically transparent window is placed in contact with tissue, the tissue may also be disposed within a depth of field of the imaging device. In some embodiments, the optically transparent window may be substantially flat, and may be configured to flatten the tissue. The inventors have also appreciated the benefits of an optically transparent window that may be the distalmost component of the imaging device that is in an optical path of the imaging device. In some embodiments, at least one flexible barrier secured to the optically transparent window may be disposed at a periphery of the optically transparent window. According to such embodiments, the at least one flexible barrier may not be disposed in an optical path of the imaging device. Of course, in other embodiments the flexible barrier may be optically transparent and may be disposed in an optical path of the imaging device, as the present disclosure is not so limited.

In addition to the above, the inventors have appreciated the benefits of a barrier that facilitates handling of a handheld imaging device. In some cases, conventional barriers for imaging devices are bulky, difficult to handle, and/or obstruct the user's ability to manipulate the imaging device. Accordingly, the inventors have appreciated the benefits of a form fitting sterile barrier that may conform to at least a portion of a handheld imaging device while still permitting the barrier to be reliably and simply deployed to cover the imaging device. In particular, the inventors have appreciated the benefits of a dual-layer sterile barrier including a first flexible barrier configured to cover an imaging device and an associated cable, and a second flexible barrier configured to cover a portion of the first flexible barrier disposed over the imaging device. The second barrier may have one or more dimensions that are smaller than the first barrier such that the second barrier may compress and/or fold the first flexible barrier in areas manipulated by a user. In this manner, the second flexible barrier may provide a form fitting barrier around a portion of the imaging device handled by a user.

In some embodiments, a barrier for an imaging device includes a cap (e.g., including an optically transparent window) configured to be removably attached to a distal portion of the imaging device. The barrier may also include a first flexible barrier secured to the cap configured to cover portions of the imaging device located proximal relative to the cap, and a second flexible barrier secured to the cap disposed over at least a portion of the first flexible barrier. The first flexible barrier may have a first maximum transverse dimension (e.g., diameter) and a first maximum longitudinal dimension (e.g., length). The second flexible barrier may have a second maximum transverse dimension (e.g., diameter) and a second maximum longitudinal dimension (e.g., length). In some embodiments the first maximum transverse dimension may be greater than the second maximum transverse dimension. For example, the first flexible barrier may have a greater diameter than a diameter of the second flexible barrier. Accordingly, when the second flexible barrier is disposed over at least a portion of the first flexible barrier, the second flexible barrier may compress and/or fold the first flexible barrier. Such an arrangement may allow the first flexible barrier to be deployed more easily, as the additional size in the maximum transverse dimension may facilitate deployment of the first flexible barrier. Once the first flexible barrier is deployed, the second flexible barrier may be deployed over the first flexible barrier. In some embodiments the first maximum longitudinal dimension may be greater than the second maximum longitudinal dimension. For example, the first flexible barrier may have a greater length than a length of the second flexible barrier. Accordingly, the second flexible barrier may cover only a portion of the first flexible barrier, such that the first flexible barrier extends past the second flexible barrier. In some embodiments, the second flexible barrier may be configured to cover an imaging device, but not cover an associated cable. In this embodiment, the first flexible barrier may be configured to cover both the imaging device and the associated cable.

According to exemplary embodiments described herein, a flexible barrier may have a maximum transverse dimension and a maximum longitudinal dimension. In some embodiments, a flexible barrier may have a maximum transverse dimension that is constant throughout the maximum longitudinal dimension. For example, a diameter of a flexible barrier may be constant throughout a length of the flexible barrier. In other embodiments, a transverse dimension of a flexible barrier may vary throughout the maximum longitudinal dimension. In some such embodiments, the transverse dimension may peak at the maximum transverse dimension. In some embodiments, a flexible barrier may have a first portion having a first maximum transverse dimension and a second portion having a second maximum transverse dimension, where the first and second maximum transverse dimensions are different. For instance, a maximum transverse dimension may increase along at least a portion of a length of a barrier extending away from the associated window and/or cap. In some embodiments, a flexible barrier may have an average maximum transverse dimension. In some embodiments where a first flexible barrier and a second flexible barrier are employed, an average maximum transverse barrier of the first flexible barrier may be greater than an average maximum transverse barrier of the second flexible barrier. In some embodiments where a first flexible barrier and a second flexible barrier are employed, a maximum transverse dimension of an imaging device covering portion of the first flexible barrier may be greater than a maximum transverse dimension of an imaging device covering portion of the second flexible barrier. Of course, a flexible barrier may have any suitable dimensions, as the present disclosure is not so limited.

In some embodiments, a flexible barrier may be formed as a film of a suitable material. In some embodiments, a flexible barrier may be formed of polyurethane. In other embodiments, a flexible layer may be formed of polyisoprene. Other materials that may be employed for a flexible barrier include, but are not limited to, low-density polyethylene (LDPE), a mixture of polyurethane and LDPE, latex, rubber, and/or other elastic polymer materials. Of course, any suitable material may be employed for a flexible barrier, as the present disclosure is not so limited.

In addition to the above, the inventors have appreciated that a frictional interface between a first flexible barrier and a second flexible barrier may facilitate manipulation of an imaging device covered by the first flexible barrier and the second flexible barrier. That is, in some cases, a low friction interface between the first flexible barrier and second flexible barrier may interfere with manipulation of the imaging device. Accordingly, the inventors have appreciated the benefits of a first flexible barrier formed of a first material, and a second flexible barrier formed of a second material. The first material and second material may be different, such that the coefficient of friction between the two materials is increased relative to a coefficient of friction between two barriers of the same material. In some embodiments, a first flexible barrier may be formed of polyurethane, and a second flexible barrier may be formed of polyisoprene. The polyisoprene may have a greater coefficient of friction with polyurethane than between two layers of polyurethane, thereby facilitating handling an imaging device covered by the first and second flexible barriers. Of course, any suitable material may be employed for a first flexible barrier and a second flexible barrier, including the same material, as the present disclosure is not so limited.

In addition to the above, the inventors have appreciated that different material properties between a first flexible barrier and a second flexible barrier may facilitate deployment of a barrier and manipulation of an imaging device covered by the first flexible barrier and the second flexible barrier. In some embodiments, a first flexible barrier may be formed of a material configured to provide a durable fluid barrier between an imaging device and a patient. For example, a first flexible barrier may be formed of a material such as polyurethane that is resistant to pinhole leaks. In some embodiments, a second flexible barrier configured to cover at least a portion of a first flexible barrier may be formed of a stretchable material. For example, a second flexible barrier may be formed of polyisoprene or another stretchable polymer material. Such an arrangement may allow the second flexible barrier to adjust to the form of an underlying imaging device. That is, the second flexible barrier may stretch to fit the form of the imaging device tightly. Of course, any suitable material may be employed for a first flexible barrier and a second flexible barrier, including the same material, as the present disclosure is not so limited.

In some embodiments, a barrier including a flexible barrier may include one or more bands configured to compress the flexible barrier against the imaging device. In some embodiments, the one or more bands may be one or more stretchable elastic bands configured to compress the flexible barrier against an imaging device. In some embodiments, the one or more elastic bands may be one or more rubber bands. In some embodiments, the one or more bands may be one or more tightenable bands. For example, in some embodiments, the one or more tightenable bands may be one or more zip-ties. In some embodiments, the one or more elastic bands may be configured to resist relative movement between the flexible barrier and the imaging device. In some embodiments where a first flexible barrier and a second flexible barrier are employed, the one or more bands may be configured to resist relative movement between the first flexible barrier and the second flexible barrier. In some embodiments, the one or more bands may include two bands. Of course, any suitable number of bands may be employed, as the present disclosure is not so limited.

In some embodiments, a method of installing a barrier on an imaging device includes removably attaching an optically transparent window and/or cap to a distal portion of the imaging device. In some embodiments, removably attaching the window and/or cap to the distal portion includes engaging the distal portion with one or more resilient latches of the optically transparent window and/or cap (e.g., in a snap fit). Of course, other connections are contemplated as described herein, as the present disclosure is not so limited. In some embodiments, removably attaching the window and/or cap to the distal portion includes covering the distal portion of the imaging device with the optically transparent window and/or cap. The method may also include covering portions of the imaging device located proximal relative to the optically transparent window and/or cap with a flexible barrier secured to the optically transparent window and/or cap. In some embodiments, covering portions of the imaging device with the flexible barrier may include unrolling the flexible barrier over the imaging device. In some embodiments, covering portions of the imaging device with the flexible barrier may include unfolding the flexible barrier over the imaging device. In some embodiments, the method may further include covering at least a portion of the flexible barrier with a second flexible barrier secured to the optically transparent window and/or cap. In some embodiments, covering at least a portion of the flexible barrier with the second flexible barrier includes stretching the second flexible barrier over the first flexible barrier. In some embodiments, covering at least a portion of the flexible barrier with the second flexible barrier includes unrolling the second flexible barrier over at least a portion of the first flexible barrier. In some embodiments, the method may include positioning at least one elastic band around the first flexible barrier and the second flexible barrier.

In some embodiments, a method of installing a barrier for an imaging device includes obtaining an optically transparent window within a housing with at least one resilient protrusion. The method may also include positioning the optically transparent window within the housing with the at least one resilient protrusion. In some embodiments, the at least one resilient protrusion where the at least one resilient protrusion is a plurality of resilient protrusions that may surround a periphery of the optically transparent window. In some embodiments, the method further includes resisting lateral movement of the optically transparent window within the housing with the at least one resilient protrusion. The method may also include inserting a distal portion of the imaging device into the housing to removably attach the optically transparent window to the distal portion of the imaging device. In some embodiments, a first threshold force may be applied to the optically transparent window to attach the window to the distal portion, for example, with a snap fit connection. In some embodiments, moving the distal portion within a threshold distance of the optically transparent window may attach the window to the distal portion, for example, with a magnetic connection. Of course, any suitable connection may be employed, as the present disclosure is not so limited. The method may also include deflecting the at least one resilient protrusion to release the optically transparent window from the housing. In some embodiments, deflecting the at least one resilient protrusion may include elastically deforming the at least one resilient protrusion. In some embodiments, deflecting the at least one resilient protrusion may include plastically deforming the at least one resilient protrusion. The method may also include deflecting the at least one resilient protrusion to allow the imaging device to pass through the housing. In some embodiments, deflecting the at least one resilient protrusion to allow the imaging device to pass through the housing includes covering the imaging device with a flexible barrier.

In some embodiments, a method of manufacturing a barrier for an imaging device includes positioning a first flexible barrier between a window and a ring. The method may also include securing the window to the ring to secure the flexible barrier to the window. The flexible barrier may be captured between the window and the ring, for example, in a press fit between the window and the ring. The method may also include cutting a portion of the flexible barrier covering a distal side (e.g., a distal end) of the window. Cutting the flexible barrier may uncover the distal end of the window, such that the flexible barrier is disposed at a periphery of the window. The method may also include removing cut portions of the flexible barrier from the window. In some embodiments, the method may also include positioning the window and flexible barrier within a housing. In some embodiments, a second flexible barrier may be secured to the optically transparent window between the ring and the first flexible barrier. Accordingly, the second flexible barrier may be proximate the ring, such that the second flexible barrier is disposed outside of the first flexible barrier.

According to exemplary embodiments described herein, a handheld medical imaging device may be employed to detect the presence of abnormal tissue with an appropriate imaging agent. In some embodiments, the medical imaging device may provide sufficient illumination of an excitation wavelength of the imaging agent to generate a fluorescence signal from the imaging agent that exceeds instrument noise of the imaging device. In some embodiments, the illumination provided by the medical imaging device may also result in an autofluorescence signal from healthy tissue. The medical imaging device may also detect abnormal tissue at sizes ranging from centimeters to sizes on the order of 10 micrometers to tens of micrometers. Other size scales are also possible. As described in more detail below, in some embodiments, it may be desirable for the medical imaging device to be able to image a large field of view in real-time and/or be relatively insensitive to human motions inherent in a handheld device as well as natural motions of a patient involved in certain types of surgery such as breast cancer and lung cancer surgeries. The imaging device may either be used for imaging surgical beds, such as tumor beds, or it may be used for imaging already excised tissue as the disclosure is not so limited.

In one embodiment, a medical imaging device may include a rigid imaging tip including a distal end defining a focal plane at a fixed distance from an optically associated photosensitive detector. For example, a distally extending member may define at its distal end a focal plane of the photosensitive detector. Depending on the embodiment, optics associated with the photosensitive detector may either fix a focus of the photosensitive detector at the focal plane located at the distal end of the rigid imaging tip, or they may permit a focus of the photosensitive detector to be shifted between the focal plane located at the distal end of the rigid imaging tip and another focal plane located beyond the distal end of the rigid imaging tip. While any appropriate photosensitive detector might be used, exemplary photosensitive detectors include a charge-coupled device (CCD) detector, a complementary metal-oxide semiconductor (CMOS) detector, and an avalanche photo diode (APD). The photosensitive detector may include a plurality of pixels such that an optical axis passes from the focal plane of the rigid imaging tip to the photosensitive detector.

Depending on the embodiment, a medical imaging device can also include one or more light directing elements for selectively directing light from a light guide including an excitation wavelength of an imaging agent towards a distal end of the device while permitting emitted light including an emission wavelength of the imaging agent to be transmitted to the photosensitive detector. In one aspect, a light emitting element includes a dichroic mirror positioned to reflect light below a wavelength cutoff towards a distal end of an associated imaging tip while permitting light emitted by the imaging agent with a wavelength above the wavelength cutoff to be transmitted to the photosensitive detector. However, it should be understood that other ways of directing light towards a distal end of the device might be used including, for example, fiber optics, LEDs located within the rigid tip, and other appropriate configurations.

An imaging device may also include appropriate optics to focus light emitted from within a field of view of the device onto a photosensitive detector with a desired resolution. To provide the desired resolution, the optics may focus the emitted light using any appropriate magnification onto a photosensitive detector including a plurality of pixels. Depending on a size of the individual pixels, the optics may either provide magnification, demagnification, or no magnification as the current disclosure is not so limited. Without wishing to be bound by theory, a typical cancer cell may be on the order of approximately 15 μm across. In some embodiments, an optical magnification of the optics within a medical imaging device may be selected such that a field of view of each pixel may be equal to or greater than about 1 μm, 2 μm, 3 μm, 4 μm, 5 μm, 10 μm, 15 μm, 30 μm, or any other desired size. Additionally, the field of view of each pixel may be less than about 100 μm, 50 μm, 40 μm, 30 μm, 20 μm, 10 μm, or any other desired size scale. In one specific embodiment, the field of view per pixel may be between about 5 μm and 100 μm inclusively. In another embodiment, the field of view per pixel may be between about 5 μm and 50 μm inclusively.

In embodiments, the medical imaging device may be associated with and/or coupled to one or more light sources. For example, a first light source may be adapted and arranged to provide light including a first range of wavelengths to a light directing element that reflects light below a threshold wavelength towards a distal end of a rigid imaging tip and transmits light above the threshold wavelength. However, other ways of directing light from the one or more light sources toward the distal end of the rigid imaging tip including fiber optics and LEDs located within the device or rigid imaging tip might also be used. Regardless of how the light is directed, the first range of wavelengths may be selected such that it is below the threshold wavelength and thus will be reflected towards the distal end of the rigid imaging tip to illuminate the device's field of view. The light source may either be a constant light source or a pulsed light source depending on the particular embodiment. Additionally, the first range of wavelengths may be selected such that it corresponds to an excitation wavelength of a desired imaging agent. It should be understood that the specific wavelength will be dependent upon the particular imaging agent, optics, as well as the sensitivity of the photosensitive detector being used. However, in one embodiment, the first range of wavelengths may be between or equal to about 300 nm to 1,000 nm, 590 nm to 680 nm, 600 nm to 650 nm, 620 nm to 640 nm, or any other appropriate range of wavelengths depending on the particular imaging agent being used. Additionally, the first light source may be adapted to provide between about 10 mW/cm² to 200 mW/cm² at a desired focal plane for imaging tissue within a surgical bed, though other illumination intensities might also be used. For example, a light intensity of 50 mW/cm² to 200 mW/cm², 100 mW/cm² to 200 mW/cm², or 150 mW/cm² to 200 mW/cm² could also be used. Depending on the particular imaging agent being used, the various components of the medical imaging device may also be constructed and arranged to collect emission wavelengths from an imaging agent that are about 300 nm to 1,000 nm, 590 nm to 680 nm, 600 nm to 650 nm, 620 nm to 640 nm, or any other appropriate range of wavelengths.

An exemplary imaging agent capable of providing the desired detection depths noted above is pegulicianine (LUM015). LUM015 and its use is further described in U.S. Patent Application Publication No. 2011/0104071 and U.S. Patent Application Publication No. 2014/0301950, which are included herein by references in their entirety. Other appropriate fluorophores that might be included in an imaging agent include, but are not limited to, Cy3, Cy3.5, Cy5, Alexa 568, Alexa 546, Alexa 610, Alexa 647, ROX, TAMRA, Bodipy 576, Bodipy 581, Bodipy TR, Bodipy 630, VivoTag 645, and Texas Red. Of course, one of ordinary skill in the art will be able to select imaging agents with fluorophores suitable for a particular application.

While various combinations of optical components and light sources are described above and in reference to the figures below, it should be understood that the various optical components such as filters, dichroic mirrors, fiber optics, mirrors, prisms, and other components are not limited to being used with only the embodiments they are described in reference to. Instead, these optical components may be used in any combination with any one of the embodiments described herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

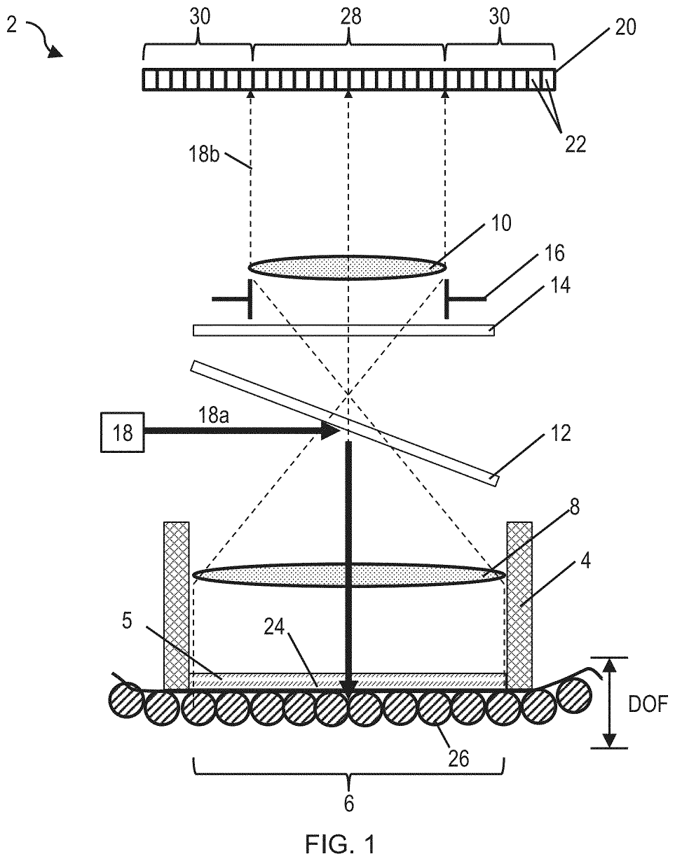

FIG. 1 depicts a schematic representation of exemplary embodiments for components of a medical imaging device 2. The medical imaging device may include a rigid imaging tip 4 at least partially defined by a distally extending member, frustoconical cylinder or other hollow structure. The rigid imaging tip 4 may be constructed and arranged to be held against tissue to fix a focal length of the medical imaging device relative to the tissue. As shown in FIG. 1, the rigid imaging tip includes an optically transparent window 5 that may be pressed into the tissue bed 24 to flatten the tissue at the fixed focal length of the medical imaging device. As depicted in FIG. 1, the rigid imaging tip 4 may also include an opening at a distal end that defines a field of view 6. The medical imaging device 2 may also include optics such as an objective lens 8, an imaging lens 10, and an aperture 16. The optics may focus light emitted from the field of view 6 onto a photosensitive detector 20 including a plurality of pixels 22. The medical imaging device may also include features such as a dichroic mirror 12 and a filter 14. While a doublet lens arrangement has been depicted in FIG. 1, it should be understood that other types of optics capable of focusing the field of view 6 onto the photosensitive detector 20 might also be used including, for example, fiber-optic bundles. Additionally, the photosensitive detector may correspond to any appropriate type of photosensitive detector configured to image or otherwise acquire a light-based signal from the field of view including photosensitive detectors such as a charge-coupled device (CCD), a complementary metal oxide semiconductor (CMOS) array, an avalanche photodiode (APD) array, or other appropriate detector.

As illustrated in FIG. 1, the medical imaging device may be positioned such that a distal end of the rigid imaging tip 4 may be pressed against a surgical bed 24 including one or more cells 26 which may be marked with a desired imaging agent. Instances where all, a portion, or none of the cells are marked with the imaging agent are contemplated. Pressing the rigid tip against the surgical bed may prevent out of plane and lateral tissue motion, which may allow for the use of collection optics with larger f numbers and consequently, larger collection efficiencies, smaller blur radii, and smaller depth of field. Additionally, pressing the rigid imaging tip 4 against the surgical bed may provide a fixed focal length between the tissue bed 24 and photosensitive detector 20. In some embodiments, the rigid imaging tip may have a length such that the distal end of the rigid imaging tip is also located at a focal plane of the photosensitive detector 20 in at least one mode of operation (e.g., when the photosensitive detector is focused on a fixed focal plane defined by the window 5). In some such embodiments, in at least one mode of operation the medical imaging device may have a fixed focal length between the tissue bed 24 and the photosensitive detector 20 as the tissue bed is pressed against the window 5. As shown in FIG. 1, the window 5 may be flat, such that the window flattens the tissue bed 24 into alignment with the distal end of the rigid imaging tip. In some embodiments, the medical imaging device may include a variable focus. According to such embodiments, in at least one mode of operation the focal plane may be adjustable, such that the focus may be set by a user based on the window 5 and tissue bed 24. For example, prior to use of the medical imaging device, the focal plane may be aligned with the window 5, or a position based at least in part on the window. As shown in FIG. 1, pressing the rigid imaging tip against the surgical bed may position the surgical bed 24 and the cells 26 contained therein within a predetermined distance (e.g., within a depth of field (DOF) of the imaging device) of the focal plane of the imaging device.

In some embodiments, it may be desirable to maintain a fixed distance between a distal end of the rigid imaging tip and the photosensitive detector. This may help to maintain the focus of tissue located within the focal plane defined by the distal end of the rigid imaging tip. Therefore, the rigid imaging tip may be adapted to resist deflection and/or deformation when pressed against a surgical bed such that tissue located within the focal plane defined by the distal end of the rigid imaging tip is maintained in focus.

During use, the medical imaging device may be associated with a light source 18 that directs light 18a with a first range of wavelengths towards the dichroic mirror 12. The first range of wavelengths may correspond to an excitation wavelength of a desired imaging agent. In some instances, the light source 18 may include appropriate components to collimate the light 18a. The light source 18 might also include one or more filters to provide a desired wavelength, or spectrum of wavelengths, while filtering out wavelengths like those detected by the photosensitive detector 20. In some embodiments, the dichroic mirror 12 may have a cutoff wavelength that is greater than the first range of wavelengths. Thus, the dichroic mirror 12 may reflect the incident light 18a towards a distal end of the rigid imaging tip 4 and onto the surgical bed 24. When the one or more cells 26 that are labeled with a desired imaging agent are exposed to the incident light 18a, they may generate a fluorescent signal 18b that is directed towards the photosensitive detector 20. The fluorescent signal may have a wavelength that is greater than the cutoff wavelength of the dichroic mirror 12. Therefore, the fluorescent signal 18b may pass through the dichroic mirror 12. The filter 14 may be a band pass filter adapted to filter out wavelengths other than the wavelength of the fluorescent signal. Alternatively, the filter 14 may permit other selected wavelengths to pass through as well. The fluorescent signal 18b may also pass through an aperture 16 to the imaging lens 10. The imaging lens 10 may focus the fluorescent signal 18b, which corresponds to light emitted from the entire field of view, onto a plurality of pixels 22 of the photosensitive detector 20. In some instances, the fluorescent signal 18b may be focused onto a first portion 28 of the photosensitive detector while second portions 30 of the photosensitive detector are not exposed to the fluorescent signal. However, in some embodiments, the fluorescent signal may be focused onto an entire surface of a photosensitive detector as the disclosure is not so limited.

Depending on the photosensitive detector used and the desired application, the one or more pixels 22 may have any desired size field of view. This may include field of views for individual pixels that are both smaller than and larger than a desired cell size. Consequently, a fluorescent signal 18b emitted from a surgical bed may be magnified or demagnified by the imaging device's optics to provide a desired field of view for each pixel 22, see demagnification in FIG. 1. Additionally, in some embodiments, the optics may provide no magnification to provide a desired field of view for each pixel 22.

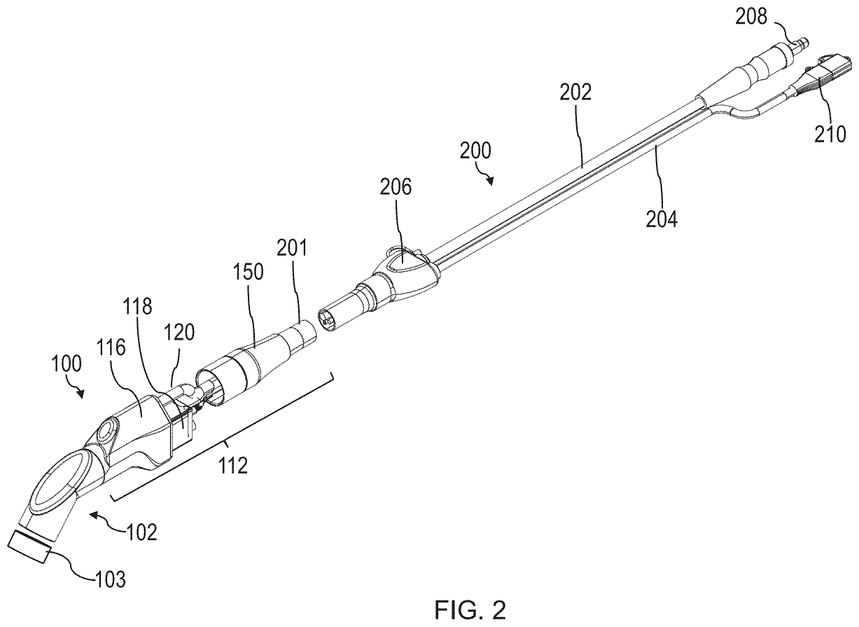

Having generally described embodiments related to a medical imaging device and an associated rigid imaging tip, specific embodiments of a medical imaging device and its components are described in more detail below with regards to FIGS. 2-4B.

FIG. 2 depicts a perspective view of a medical imaging device 100 and hybrid cable 200. As shown in FIG. 2, the imaging device 100 includes a rigid imaging tip 102 configured to be placed on tissue to image the tissue at a focal length set by a distal end of the imaging tip. The imaging device includes a body 112 that may be manipulated by a user (e.g., a surgeon). In some embodiments as shown in FIG. 2, the body of the device includes a housing 116 having a portion that functions as a handle so that the device may be hand operated. The body houses a light guide 120 and a photosensitive detector 118. The light guide 120 may be configured to illuminate the targeted tissue for imaging. In particular, the light guide 120 may be configured to provide an excitation light at a desired wavelength range that excites fluorescence of an imaging agent. As will be discussed further with reference to exemplary embodiments below, the light may pass from the light guide 120 through several reflecting surfaces, lens, filters, and/or other optical elements before reaching the imaging tip 102. The light guide 120 as shown in FIG. 2 is a fiber optic cable, which may be connected to an external light source via the hybrid cable 200. As shown in FIG. 2, the light guide 120 and the photosensitive detector 118 are attached to a housing 116. The housing 116 may house the various optical components. The housing may also include the imaging tip 102. As shown in FIG. 2, the medical imaging device includes a removable tip 103 that may be attached to the imaging tip 102. As will be discussed further below, the removable tip 103 may include a window and may be configured to engage a tissue bed to flatten the tissue bed within a depth of field of the photosensitive detector 118. The housing 116 may also provide a handling surface (e.g., a handle) for a user of the medical imaging device 100. According to some embodiments as shown in FIG. 2, the medical imaging device may also include a tapered housing portion 150 which may assist in sealing the housing 116 from fluid ingress. In some embodiments, the tapered housing portion may compress and seal a portion 201 of the hybrid cable 200 entering the body 112.

According to the embodiment of FIG. 2, the medical imaging device 100 includes a hybrid cable 200. The hybrid cable may function to connect the light guide 120 and the photosensitive detector 118 to an external light source, a power source and/or processor, respectively. As shown in FIG. 2, the hybrid cable includes an optical cable 202 configured to pipe light from an external light source to the light guide 120. The hybrid cable 200 also includes a detector cable 204. In some embodiments, the detector cable 204 may transmit both power and signals from the photosensitive detector in some embodiments. However, instances in which separate cables are used for power and signal transmission are also contemplated. Regardless of the specific arrangement, the detector cable 204 may connect the photosensitive detector 118 to a computing device including one or more processors configured to receive signals from the photosensitive detector. In some embodiments, the detector cable may employ a standardized protocol for data and power, such as USB 2.0, USB 3.0, USB-C, or any other suitable protocol. As sown in FIG. 2, the hybrid cable includes a proximal connector 206 which receives both the optical cable 202 and the detector cable 204. In some embodiments, the proximal cable is configured to provide a waterproof seal between the optical cable and the detector cable. The hybrid cable also includes an optical connector 208 configured to connect to an external light source. The hybrid cable also includes a detector connector 210 configured to connect the detector to an external device (e.g., a computing device). Of course, while a wired medical imaging device 100 is shown including a hybrid cable 200 in the embodiment of FIG. 2, in other embodiments data may be transmitted wirelessly to an external device (e.g., a computing device). For example, the medical imaging device 100 may include a wireless transmitter or transceiver configured to send or receive information from an external device (e.g., a computing device). In some embodiments, a medical imaging device 100 may be wired to a light source and power source but may transmit information wirelessly to an external device having one or more processors. Of course, any suitable combination of wired and wireless connections may be employed, as the present disclosure is not so limited.

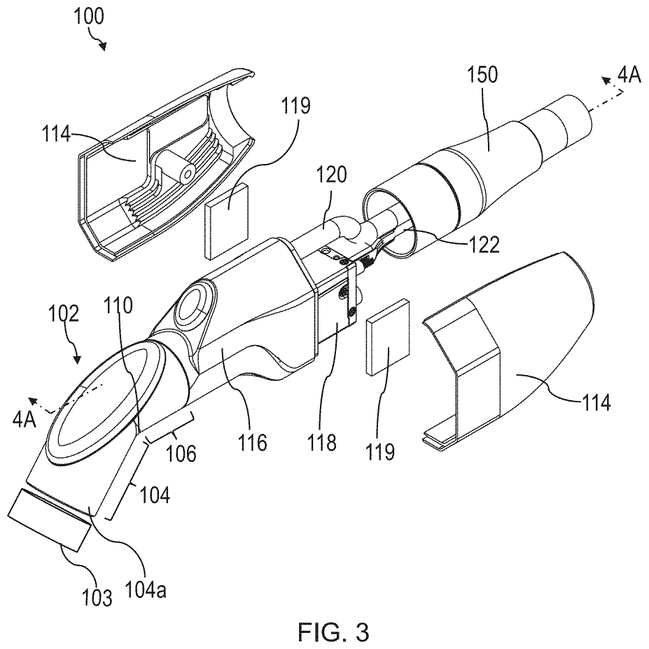

FIG. 3 depicts a partially exploded view of a medical imaging device 100 including a distally extending rigid imaging tip 102. The rigid imaging tip 102 may include a distal portion 104 and a proximal portion 106. A distal end 104a of the rigid imaging tip located on the distal portion 104 may at least partly define a field of view for the imaging device. In some embodiments, the proximal portion 106 may be constructed to either be detachably or permanently connected to a housing 116 of the imaging device. In some embodiments, the rigid imaging tip may also be made from materials that are compatible with typical sterilization techniques such as various steam, heat, chemical, and radiation sterilization techniques.

As shown in FIG. 3, the medical imaging device 100 includes a removable tip 103 configured to be removably attached to the distal end 104a of the rigid imaging tip 102. The removable tip may be configured to protect the rigid imaging tip during use of the device with a tissue bed. In some embodiments, the removable tip 103 may include one or more optically transparent windows configured to allow light to pass through the removable tip. In some embodiments, the removable tip may be configured to be pressed against a tissue bed to flatten the tissue within a depth of field of a photosensitive detector 118. In some embodiments, the connection between the rigid imaging tip 102 and the removable tip 103 may include, for example, a snap on, screw on, suction, magnetic connection, and/or any other appropriate type of connection. This may provide multiple benefits including, for example, easily and quickly changing a rigid imaging tip during a surgical procedure as well as enabling the rigid imaging tip to be removed and sterilized. In some embodiments, the removable tip 103 may be removed from the medical imaging device after use.

In some embodiments as shown in FIG. 3, the housing 116 of the medical imaging device 100 may include a light guide covering portion 114. As shown in FIG. 3, the housing 116 is configured to mount the photosensitive detector 118 to the medical imaging device. The light guide covering portion 114 houses thermal pads 119 configured to absorb heat from the photosensitive detector. In some embodiments, the light guide covering portion 114 may be configured to cover the light guide 120 and the photosensitive detector 118. In some embodiments, the photosensitive detector 118 may include an appropriate data output 122 for outputting data to an external device (e.g., a computing device). In some embodiments, the data output may include a detector cable, as described previously with reference to FIG. 2. Additionally, in some embodiments, the photosensitive detector may include a power input. In some embodiments, the power input may include a detector cable, as described previously with reference to FIG. 2. In some embodiments, the data output 122 may include an integrated power input to the photosensitive detector 118, for example, in the form of a detector cable (see FIG. 2, for example). In some embodiments, one or more light guides 120 associated with one or more separate light sources, not depicted, may be covered by the light guide covering portion 114. As discussed previously the light guide 120 may provide light including at least a first range of excitation wavelengths to the medical imaging device 100. According to the embodiment of FIG. 3, the medical imaging device includes a tapered housing portion 150 configured to compress and seal any cable(s) entering the housing 116.

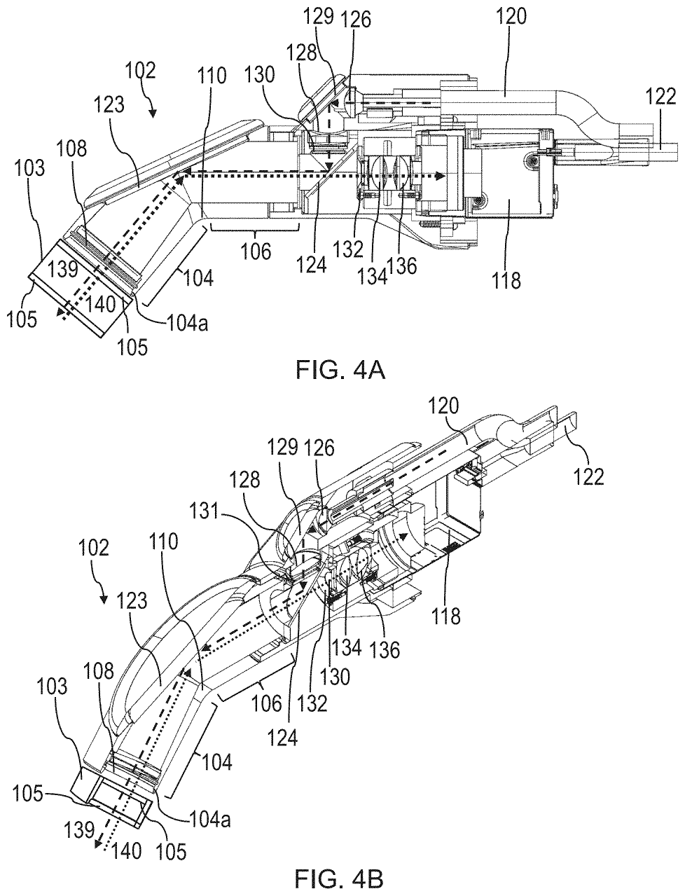

FIGS. 4A-4B depict cross sectional views of the medical imaging device of FIG. 3 taken along line 4A-4A. The cross sections of FIGS. 4A-4B depict the optical arrangement of the medical imaging device. As shown in FIGS. 4A-4B, the medical imaging device includes a rigid imaging tip 102 corresponding to a member distally extending from the housing 116 with an optically transparent or hollow interior. A distal end 104a of the rigid imaging tip 102 may define a focal plane located at a fixed distance relative to the optically coupled photosensitive detector 118 located on a proximal portion of the medical imaging device. In one embodiment, the optics coupling the rigid imaging tip and the photosensitive detector may include an objective lens 134 and an imaging lens 136 located between the rigid imaging tip and the photosensitive detector. The objective and imaging lenses 134 and 136 may focus light emitted from within a field of view of the rigid imaging tip onto a surface of the photosensitive detector 118 including a plurality of pixels. A magnification or demagnification provided by the combined objective and imaging lenses 134 and 136 may be selected to provide a desired field of view for each pixel.

As shown in FIGS. 4A-4B, the medical imaging device 100 may also include one or more dichroic mirrors 124 located between the photosensitive detector 118 and a distal end 104a of the rigid imaging tip. The dichroic mirror 124 may be adapted to reflect light below a cutoff wavelength towards the distal end of the rigid imaging tip and transmit light above the cutoff wavelength towards the photosensitive detector 118. In the current embodiment, the cutoff wavelength may be greater than an excitation wavelength of a desired imaging agent and less than an emission wavelength of the imaging agent. While any appropriate structure might be used for the dichroic mirror, in one embodiment, the medical imaging device includes a single dichroic mirror along an optical path of the medical imaging device.

In some embodiments as shown in FIGS. 4A-4B, the medical imaging device 100 may include one or more filters 130 located between the dichroic mirror 124 and the photosensitive detector 118. The one or more filters 130 may be adapted to permit light emitted from an imaging agent to pass onto the photosensitive detector while blocking light corresponding to excitation wavelengths of the imaging agent. Depending on the embodiment, the one or more filters may either permit a broad spectrum of wavelengths to pass or they may only permit the desired excitation wavelength, or a narrow band surrounding that wavelength, to pass as the disclosure is not so limited.

In some embodiments as shown in FIG. 4A-4B, an aperture stop 132 including an appropriately sized aperture may also be located between the rigid imaging tip 102 and the photosensitive detector 118. More specifically, the aperture stop 132 may be located between the dichroic mirror 124 and the imaging lens 136. Depending on the embodiment, the aperture may have an aperture diameter selected to provide a desired f number, depth of field, and/or reduction in lens aberrations. Appropriate aperture diameters may range from about 5 mm to 15 mm inclusively which may provide an image side f number between about 3 to 3.5 inclusively. However, other appropriate aperture diameters and f numbers are also contemplated.

During use of the medical imaging device 100, the light guide 120 may receive light from an associated light source. The light guide 120 may be any appropriate structure including, for example, fiber-optic cables used to transmit light from the associated light source to the medical imaging device. According to the embodiment of FIGS. 4A-4B, the light guide 120 is configured to extend in a direction that is parallel to a longitudinal axis of a portion of the medical imaging device the light guide extends through. Accordingly, as shown in FIGS. 4A-4B, the light guide 120 is orientated parallel to the direction of imaging of the photosensitive detector 118 along an associated portion of the optical path though other orientations of these components may also be used as the disclosure is not so limited. In some embodiments, the light guide 120 may be associated with optics such as an aspheric lens 126 disposed on a distal end of the depicted optical fiber bundle of the light guide 120 to help collimate light directed towards the dichroic mirror 124. As shown in FIGS. 4A-4B, the light guide may also include an additional collimating lens to further collimate light toward the dichroic mirror 124. The light guide 120 may also be optically coupled with one or more filters 131 disposed between the light guide and the dichroic mirror in order to provide a desired wavelength, or a spectrum of wavelengths to the dichroic mirror 124 and ultimately the rigid imaging tip 102. This wavelength, or spectrum of wavelengths, may correspond to one or more excitation wavelengths of a desired imaging agent used to mark abnormal tissue for imaging purposes. Depending on the embodiment, the light guide 120 may either be associated with a single light source, or it may be associated with multiple light sources. Alternatively, multiple light inputs may be coupled to the medical imaging device to provide connections to multiple light sources as the current disclosure is not so limited.

According to the embodiment of FIGS. 4A-4B, as the light guide 120 is oriented parallel to a longitudinal axis of the of the medical imaging device, the dichroic mirror 124 is not in a direct optical path of the light guide. Accordingly, as shown in FIGS. 4A-4B, the medical imaging device may include a light guide mirror 129 configured to redirect the light from the light guide 120 towards the dichroic mirror 124. That is, the light guide mirror 129 reflects the light from the light guide approximately 90 degrees toward the dichroic mirror 124. In some embodiments as shown in FIGS. 4A-4B, the light guide mirror is disposed between the aspheric lens 126 and the collimating lens 128, though other arrangements are contemplated, and the disclosure is not so limited. The path of light provided by the light guide is shown by light guide path 139, which is discussed further below. While a mirror is employed in the embodiment of FIGS. 4A-4B, in other embodiments other light bending elements may be employed, including, but not limited to, prisms, fiber optics, etc., as the present disclosure is not so limited.

It should be understood that the above components may be provided in any desired arrangement. Additionally, a medical imaging device may only include some of the above noted components and/or it may include additional components. However, regardless of the specific features included, an optical path 140 of a medical imaging device may pass from a distal end 104a of a rigid imaging tip 102 to a photosensitive detector 118. For example, light emitted from within a field of view may travel along an optical path 140 passing through the distal end 104a as well as the distal portion 104 and proximal portion 106 of the rigid imaging tip. The optical path may also pass through the housing 116 including various optics to the photosensitive detector 118.

According to the embodiment of FIGS. 4A-4B, a medical imaging device 100 includes a rigid imaging tip 102 with a distal portion 104 and a proximal portion 106. The distal portion 104 may include a distal end 104a including an opening optically coupled with a photosensitive detector 118. The rigid imaging tip includes a window 108 integrated with the distal end 104a of the rigid imaging tip. The window 108 may be transparent to both the excitation wavelengths provided by an associated light source as well as wavelengths emitted from a desired imaging agent. While any appropriate shape might be used depending on the particular optics and algorithms used, in one embodiment, the window 108 may have a flat shape to facilitate placing tissue at a desired focal plane when it is pressed against a surgical bed. Additionally, as shown in the embodiment of FIGS. 4A-4B, the medical imaging device 100 includes a removable tip 103 configured to be removably attached to the distal end 104a of the rigid imaging tip 102. The removable tip may be configured to protect the rigid imaging tip during use of the device with a tissue bed. The removable tip 103 includes two optically transparent windows 105 configured to allow light to pass through the removable tip. In particular, the windows 105 may be transparent to both the excitation wavelengths provided by an associated light source as well as wavelengths emitted from a desired imaging agent. Of course, while two windows are shown in the embodiment of FIGS. 4A-4B, in other embodiments any suitable number of windows may be employed, as the present disclosure is not so limited. In some embodiments, the removable tip 103 may be configured to be pressed against a tissue bed to flatten the tissue within a depth of field of the photosensitive detector 118. For example, one of the windows 105 may be pressed against the tissue to flatten the tissue against the window. In some embodiments a focal plane of the photosensitive detector may be aligned with a distal window 105 of the removable tip 103, such that tissue pressed against the distal window is within a depth of field of the photosensitive detector. In some embodiments, the connection between the rigid imaging tip 102 and the removable tip 103 may include, for example, a snap on, screw on, suction, magnetic connection, and/or any other appropriate type of connection.

In some embodiments as shown in FIGS. 4A-4B, the rigid imaging tip 102 includes a bend 110 to facilitate access of a medical imaging device into a surgical site. For example, a distal portion 104 of the rigid imaging tip may be angled relative to a proximal portion 106 of the rigid imaging tip. Any appropriate angle between the proximal and distal portions to facilitate access to a desired surgical site might be used. However, in one embodiment, an angle between the proximal and distal portions may be between about 25° to 65°. For example, a rigid imaging tip may have an angle that is equal to about 45°. In embodiments including an angled distal portion, the rigid imaging tip 102 includes a mirror 123 adapted to bend an optical path 140 and light guide path 139 through the bent rigid imaging tip. The mirror may be positioned at the bend 110 of the rigid imaging tip, such that light traveling through the proximal portion 106 is reflected through the distal portion 104. Likewise, light traveling through the distal portion 104 is reflected by the mirror through the proximal portion 106. In this manner the mirror provides a reflective surface allowing for the transmission of both excitation light and light emitted from a desired imaging agent to travel through the rigid imaging tip 102. It should be understood that even though a bent configuration with a mirror 123 is shown in the exemplary embodiment of FIGS. 4A-4B, one or more other light bending components (e.g., prisms, fiber optics, etc.) may be employed, as the present disclosure is not so limited. Additionally, in some embodiments, a straight imaging tip may be employed without any mirror, as the present disclosure is not so limited.

According to the embodiment of FIGS. 4A-4B, the light guide path 139 and optical path 140 are substantially parallel along at least a portion of a length of the imaging device. The optical path 140 originates at the distal end 104a, reflects off the mirror 123 and proceeds through the dichroic mirror 124 to the photosensitive detector. The light guide path originates at the light guide 120, reflects off the light guide mirror 129, reflects off the dichroic mirror 124 toward the rigid imaging tip 102, and finally reflects off the mirror 123 and exits the distal end 104a of the rigid distal tip. Accordingly, the light guide path 139 and optical path 140 are parallel from the dichroic mirror 124 through the distal end 104a of the rigid imaging tip. In some embodiments, a portion of the optical path 140 and a portion of the light guide path 139 are coincident along a length of the imaging device between the dichroic mirror 124 and the distal end 104a. Of course, any suitable optical path and light guide path may be employed in a medical imaging device, as the present disclosure is not so limited.

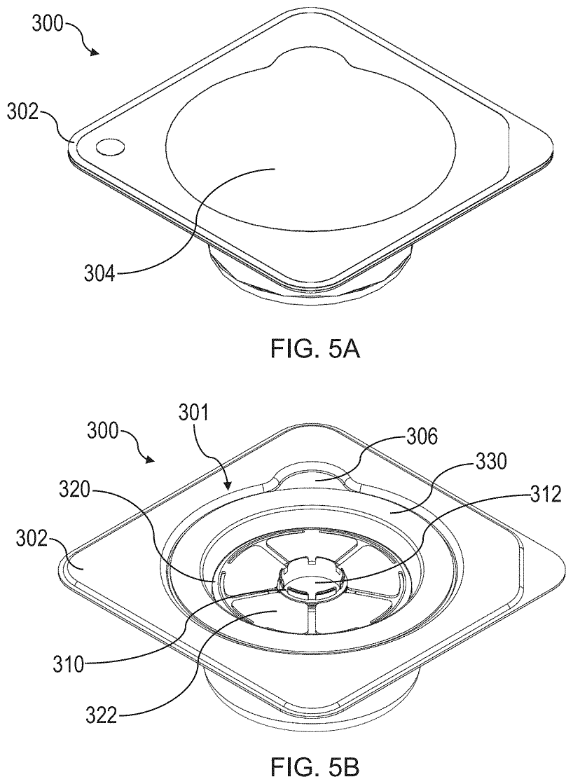

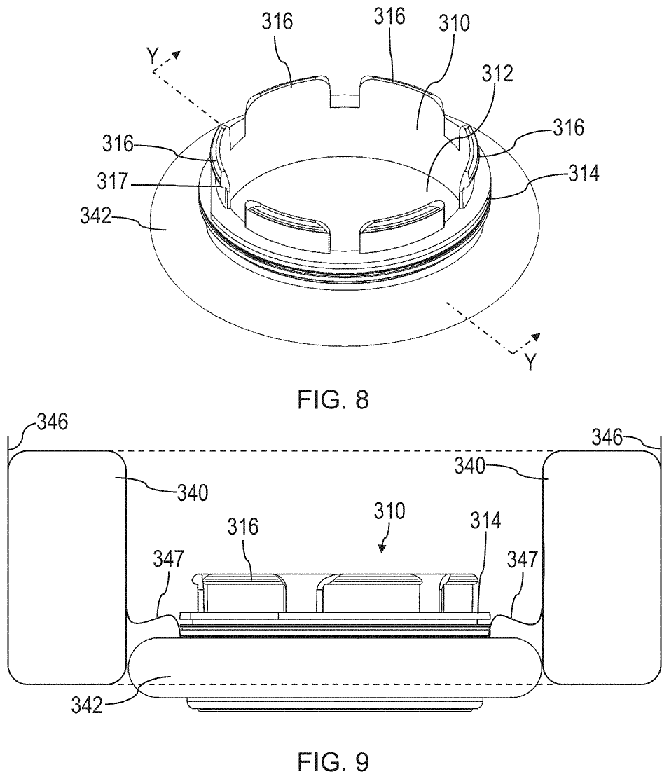

FIG. 5A is a perspective view of one embodiment of a sterile barrier package 300. According to the embodiment of FIG. 5A, the sterile barrier package is configured to contain a barrier for an imaging device. The package 300 may maintain the sterility of the barrier until the barrier is ready for use. The package 300 includes a shell 302 configured to retain and support the barrier, as will be discussed further in reference to FIG. 5B. As shown in FIG. 5A, the package 300 includes a lid 304 disposed on the shell 302. In some embodiments, the lid 304 may be formed of polyethylene fibers or another suitable material. In some embodiments the lid 304 may include a protective layer (e.g., a card) configured to resist abrasion or puncturing from components of barrier assembly disposed in the shell 302. The protective layer may be disposed on an interior surface of the lid 304 facing components of a sterile barrier assembly disposed within the shell 302. For example, the protective layer may resist abrasion or puncturing from one or more resilient latches that may contact the lid 304 (see, e.g., one or more resilient latches 316 of exemplary FIG. 8). In some embodiments, the protective layer may be formed of a semi-rigid material (e.g., plastic, cardboard, etc.), though any suitable material may be employed, as the present disclosure is not so limited.

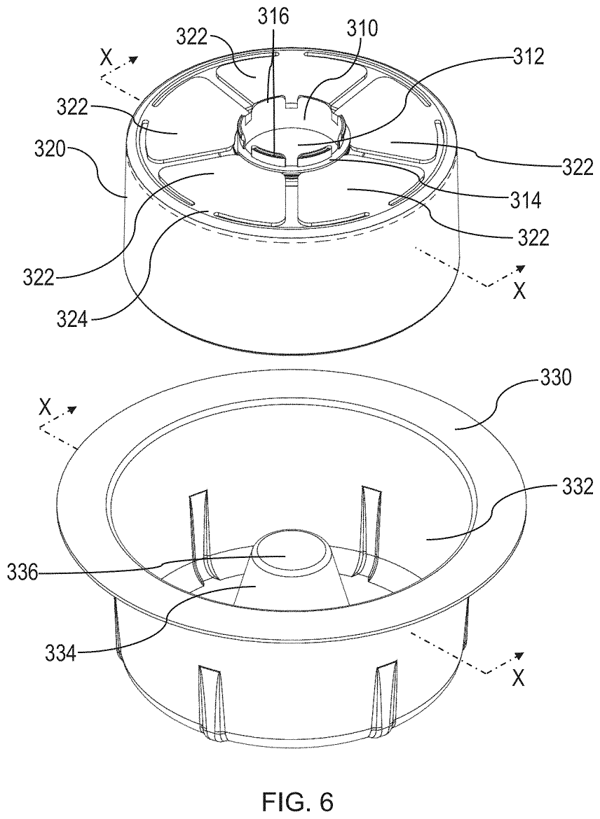

FIG. 5B is a perspective view of the sterile barrier package of FIG. 5A with the lid removed. As shown in FIG. 5B, the sterile barrier package contains a barrier assembly 301 disposed in the shell 302. The barrier assembly includes a cap 310 and a housing 320. According to the embodiment of FIG. 5B, the cap 310 includes an optically transparent window 312. The cap 310 is configured to be releasably attached to a distal portion of an imaging device. The housing 320 includes a plurality of resilient protrusions 322, which in the embodiment of FIG. 5B are configured as a plurality of resilient fins extending radially inwards towards and contacting the cap 310. Thus, the plurality of resilient protrusions at least partially surrounds a periphery of the cap 310 and positions the cap within the housing 320. Additionally, the plurality of resilient protrusions is configured to resist lateral movement of the cap within the housing. In some embodiments as shown in FIG. 5B, the barrier assembly includes a tray 330 disposed in the shell 302. The tray 330 is configured to support the housing 320. The tray 330 is also configured to allow a user to remove the housing 320 and the cap 310 without directly contacting the housing or the cap. The tray may be removed from the shell via a cutout 306 formed in the shell. The functionality of the cap 310, the plurality of resilient protrusions 322, and the tray 330 will be discussed further with reference to the exemplary embodiments of FIGS. 6-11D.

FIG. 6 is a partially exploded view of one embodiment of a sterile barrier assembly including a tray 330. As shown in the exemplary embodiment of FIG. 6, the sterile barrier assembly includes a cap 310 having an optically transparent window 312 disposed in the cap. The cap includes a peripheral shelf 314 which is configured to be engaged by a plurality of resilient protrusions 322. In some embodiments as shown in FIG. 6, the peripheral shelf abuts the plurality of resilient protrusions such that the cap 310 may not be moved without deflecting the plurality of resilient protrusions (e.g., by applying a force greater than a threshold force to the cap to remove the cap from the resilient protrusions). In some embodiments as shown in FIG. 6, the cap 310 includes a plurality of resilient latches 316 that are configured to engage a distal portion of an imaging device. For example, the plurality of resilient latches may engage an inner wall of the distal portion of the imaging device to releasably attach the cap to the distal portion of the imaging device. In some embodiments, the resilient latches may be configured to deflect as the resilient latches are received by the distal portion of the imaging device. In some embodiments, the resilient latches may be received in one or more receptacles formed in the distal portion of the imaging device. In some embodiments, when the cap is releasably attached to the distal portion of the imaging device, the imaging device may be employed to deflect the plurality of resilient protrusions 322 and release the cap 310. Of course, while a plurality of resilient latches 316 and protrusions are described relative to FIG. 6, any suitable number of latches and/or protrusions may be employed, including one, as the present disclosure is not so limited. Likewise, other types of connections may also be used as previously described, as the present disclosure is not so limited.

As shown in FIG. 6, the sterile barrier assembly includes a housing 320 including the plurality of resilient protrusions. According to some embodiments as shown in FIG. 6, the plurality of resilient protrusions may be integrally formed with the housing 320. In other embodiments, the resilient latches may be separate components from the housing, as the present disclosure is not so limited. In the depicted embodiment of FIG. 6, the plurality of resilient protrusions is configured as a plurality of resilient fins. The fins are connected to the remainder of the housing 320 via a narrowing portion 324. The narrowing portion 324 may have a maximum transverse dimension (e.g., width) that is less than a maximum transverse dimension of the plurality of resilient protrusions. The narrowing portion may reduce the resistance to deflection of the plurality of resilient protrusions at the narrowing portion. Accordingly, less force may be used to deflect the plurality of resilient protrusions than would be employed with no narrowing portion. In other embodiments, a resilient protrusion may have a constant transverse dimension and may not include a narrowing portion, as the present disclosure is not so limited. In some embodiments as shown in FIG. 6, the housing is annular, and is configured to position the cap 310 along a central longitudinal axis of the housing with the plurality of resilient protrusions.

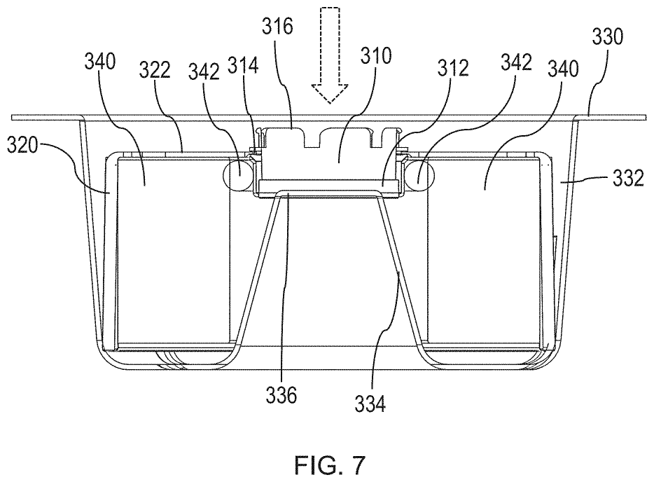

According to the embodiment of FIG. 6, the sterile barrier assembly includes a tray 330. The tray 330 may be configured to be handled by a user, such that contact with the housing 320 and cap 310 may be avoided. In this manner, the tray 330 may promote sterility of the cap 310 and the housing 320 which may ultimately be employed during a surgical procedure, whereas the tray may be disposed of. As shown in FIG. 6, the tray includes a receptacle 332 configured to receive and support the housing 320. In some embodiments as shown in FIG. 6, the tray 330 also includes a support 334 disposed along a central longitudinal axis of the tray. In the embodiment of FIG. 6, the support is configured as a post. Of course, in other embodiments, a support may have any suitable shape, including, but not limited to, a cylindrical shape, frustoconical shape, and annular shape. In some embodiments, a tray may include one or more supports, as the present disclosure is not so limited. The support 334 is configured to engage and support the cap 310. In particular, the support 334 includes a cap engagement surface 336 configured to abut the cap 310 and resist longitudinal movement of the cap in one direction. For example, if force is applied to the cap 310 in a direction toward the tray 330, the cap engagement surface 336 is configured to resist movement of the cap 310 in that direction. Accordingly, the support and cap engagement surface may facilitate the attachment of the cap to a distal portion of an imaging device. For example, the cap engagement surface 336 may allow a threshold force to be applied to the cap 310 by a distal portion of an imaging device without moving the cap 310, such that the cap may be releasably attached to the distal portion. The support 334 may also maintain a height of the cap 310 relative to the housing 320, such that the cap is easily accessible to the distal portion of the imaging device. In some embodiments, once the cap 310 is attached to the distal portion of an imaging device, the housing 320 may be removed from the tray 330. In some embodiments, the cap engagement surface 336 may be configured to engage the window 312 directly and may resist longitudinal movement of the window 312 in at least one longitudinal direction. The engagement between the cap engagement surface and the cap is shown and described further below with reference to FIG. 7.

FIG. 7 is a cross-sectional view of the sterile barrier assembly of FIG. 6 taken along lines X-X. As shown in FIG. 7, the cap 310 and the housing 320 are disposed in the tray 330. In particular, the housing 320 is received in the receptacle 332 of the tray. The cap 310 is positioned in the housing 320 by the plurality of resilient protrusions. The plurality of resilient protrusions engages a peripheral shelf 314 of the cap 310 to retain the cap in the housing until the plurality of resilient protrusions is deflected. As shown in FIG. 7, the cap 310 includes an optically transparent window 312. The optically transparent window is configured to be optically transparent to light emitted from or received by an associated imaging device. In some embodiments, the window 312 is substantially flat (e.g., has a peak-to-valley deviation less than 200 nm, 80 nm, or another suitable distance). A distal surface of the window may be configured to be disposed within a depth of field of an associated imaging device, such that tissue in contact with the distal surface of the window is also in the depth of field, when the window is selectively attached to the distal portion of the imaging device. In some embodiments, the window may be configured to be pressed against tissue to flatten the tissue against the window. In some embodiments as shown in FIG. 7, the window may form a distal portion of the cap 310. Additionally, in the embodiment of FIG. 7, the cap 310 is annular and the window extends across and closes the distal end of the cap.

As shown in FIG. 7, the cap engagement surface 336 of the support 334 is engaged with the optically transparent window 312. That is, the cap engagement surface is in contact with a distal surface of the window, such that the cap engagement surface supports the window 312 and the cap 310. The cap engagement surface is configured to resist longitudinal movement of the window and cap in a direction toward the support 334. Accordingly, if force is applied to the cap 310 in a longitudinal direction toward the tray 330 as shown by the dashed arrow, the cap engagement surface will inhibit movement of the window 312 and the cap 310 in that direction. Accordingly, a force greater than a threshold force oriented in the longitudinal direction may be applied to the cap with a distal portion of an imaging device to releasably attach the cap to the distal portion of the imaging device without removing the cap from the housing 320 because the support resists movement in that direction. As discussed previously, the plurality of resilient protrusions will resist lateral movement of the cap in the housing 320 so that a user may reliably attach the distal portion of the imaging device to the cap. Once the cap is releasably attached to the imaging device, the imaging device may be employed to deflect the plurality of resilient protrusions 322 to release the cap from the housing. For example, in some embodiments the tray 330 may be removed following the attachment of the cap to the imaging device. A distal force (e.g., in a longitudinal direction shown by the dashed arrow) greater than the threshold force may then be applied to the cap 310 with the imaging device to deflect the plurality of resilient protrusions, thereby releasing the cap since the cap is no longer supported by the support. In such an embodiment, the removal of the tray removed the support of the cap engagement surface 336, which allows the cap to move relative to the housing in the direction the cap engagement surface previously occupied.

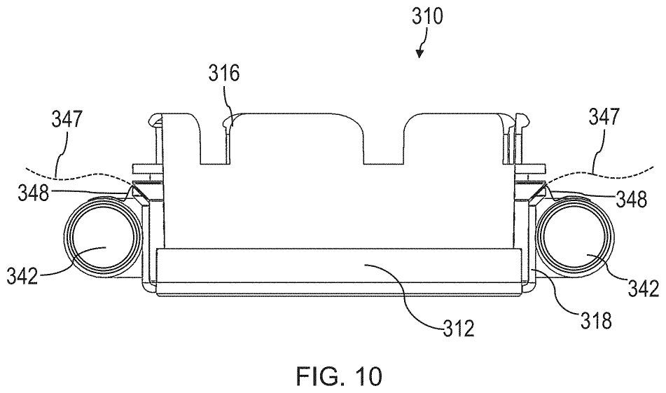

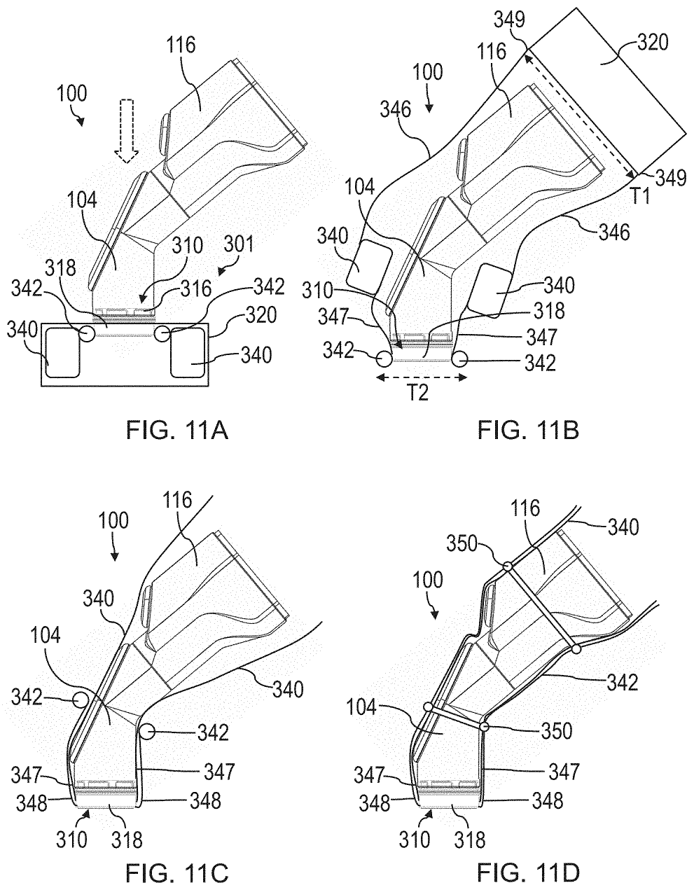

According to the embodiment of FIG. 7, the sterile barrier assembly includes a first flexible barrier 340 and a second flexible barrier 342. As shown in FIG. 7, the first flexible barrier and second flexible barrier are disposed in the housing 320 in an initial configuration. The first flexible barrier and second flexible barrier are both secured to the cap 310. An exemplary connection between a flexible barrier and the cap will be described further with reference to FIG. 10, though any suitable connection may be employed. The first flexible barrier is configured to cover portions of an imaging device and associated cable proximal the optically transparent window 312. The second flexible barrier 342 is configured to cover a portion of the first flexible barrier disposed over the imaging device. The second flexible barrier 342 may be configured to compress and/or fold the first flexible barrier, to facilitate the manipulation of the imaging device while covered by the first and second flexible barriers. In the embodiment of FIG. 7, the first flexible barrier has a maximum longitudinal dimension greater than that of a maximum longitudinal dimension of the second flexible barrier. Additionally, in the embodiment of FIG. 7, the first flexible barrier has a maximum transverse dimension greater than that of a maximum transverse dimension of the second flexible barrier. An exemplary embodiment depicting the dimensional relationship between a first flexible barrier and second flexible barrier will be described further with reference to FIGS. 11A-11D. In some embodiments, the first flexible barrier and second flexible barrier may be rolled, such that the first flexible barrier and second flexible barrier are unrolled to cover portions of an imaging device and associated cable.

FIG. 8 is a perspective view of one embodiment of a removable cap 310 showing detail of the cap 310 and a flexible barrier 342 extending out from the removable cap. As shown in FIG. 8, the cap 310 includes an optically transparent window 312. Additionally, the cap includes a peripheral shelf 314 that is configured to be engaged by one or more resilient protrusions. As shown in FIG. 8, the peripheral shelf extends around a perimeter of the cap. Additionally, the cap 310 includes a plurality of resilient latches 316. In the embodiment of FIG. 8, the cap includes six resilient latches disposed around the periphery of the cap. Each of the resilient latches is configured to move between an engaged position and a disengaged position. In the engaged position, the plurality of resilient latches is configured to engage one or more receptacles of a distal portion of an imaging device. In particular, catches 317 of each resilient latch are configured to resist removal of the cap from the distal portion of an imaging device. The resilient latches may be configured to deflect (e.g., elastically deform) between the engaged position and the disengaged position. In the disengaged position, the resilient latches may be configured to release from the distal portion of the imaging device. In some embodiments, the resilient latches may deflect to the disengaged position when received by the distal portion of the imaging device. In some embodiments, the resilient latches may deflect to the disengaged position when a threshold release force is applied to the cap to release the cap from the distal portion and allow the cap to be removed from the imaging device. As shown in FIG. 8, the flexible barrier 342 is secured to the optically transparent window 312 (e.g., via the cap 310) and is configured in a roll. The flexible barrier may be unrolled to cover at least a portion of an imaging device disposed proximal to the window 312. Of course, while a roll is employed in the embodiment of FIG. 8, in other embodiments a flexible barrier may be folded, as the present disclosure is not so limited.

FIG. 9 is a side view of the removable cap 310 of FIG. 8 including a first flexible barrier 340 and a second flexible barrier 342. For the purposes of illustration, the first flexible barrier 340 is illustrated transparently to show the other portions of the cap. The first flexible barrier and second flexible barrier may be annular. In the embodiment of FIG. 9, the first flexible barrier and second flexible barrier both have a tori shape when they are in the rolled configuration (e.g., donut shape). The first flexible barrier and second flexible barrier may be formed of a roll of a flexible film material. In some embodiments as shown in FIG. 9, a barrier may include multiple flexible barriers configured to cover portions of an imaging device. In other embodiments, a single flexible barrier may be employed (e.g., see FIG. 8), as the present disclosure is not so limited. According to the embodiment of FIG. 9, the first flexible barrier 340 is a longer and transversely larger than the second flexible barrier 342. The first flexible barrier 340 may be deployed (e.g., unrolled) over an imaging device first. The second flexible barrier 342 may be deployed (e.g., unrolled) over the first flexible barrier after the first flexible barrier is deployed. In this manner, the second flexible barrier may compress and/or fold the first flexible barrier, to conform the first and second flexible barriers to the shape of at least a portion of the imaging device. According to the embodiment of FIG. 9, the first flexible barrier may include a first end portion 346 and a second end portion 347. The second end portion may be secured to the cap 310, and the first end portion may be secured to a housing or may be independent and not secured to any other structure. The first flexible barrier may be unrolled by applying tension between the first and second end portion. In contrast, the second flexible barrier includes a single end portion that is secured to the cap 310. Accordingly, to deploy the second flexible barrier, the second flexible barrier may be unrolled by moving the second flexible barrier roll in an unrolling direction (e.g., proximal direction). Of course, any suitable deployment arrangement may be employed for a flexible barrier, including unfolding, as the present disclosure is not so limited.

FIG. 10 is a cross-sectional view of the removable cap 310 of FIG. 8 taken along line Y-Y, showing the connection between the flexible barrier 342 (and optionally an additional flexible barrier) and the cap 310. As shown in FIG. 10, the cap is configured to receive a ring 318. For example, in some embodiments, the ring may be press fit onto the cap 310. Of course, in other embodiments other mounting arrangements may be employed, including, but not limited to, set screws, fasteners, adhesives, etc. In some embodiments, the ring 318 may also be employed to secure the optically transparent window 312 to the cap 310. As shown in FIG. 10, the flexible barrier 342 includes an end portion 348. The end portion 348 may be captured between the cap 310 and the ring 318. Accordingly, when the ring is secured to the cap (e.g., via a press fit), the end portion 348 will be secured between the ring and the cap. In some embodiments, multiple flexible barriers may be secured to the cap in this manner. For example, an additional end portion 347 of another flexible barrier may be disposed between the ring and the cap. Accordingly, a plurality of flexible barriers may be secured to a cap according to the arrangement in FIG. 10. Of course, in other embodiments a flexible barrier may be secured to a cap and/or optically transparent window using any suitable arrangement, including ultrasonic welding or adhesives, as the preset disclosure is not so limited. In some embodiments, a flexible barrier having a radially outermost end portion may be configured to cover at least portions of flexible barriers having end portions disposed radially inward of the radially outermost end portion. For example, in the embodiment of FIG. 10, the flexible barrier 342 having an end portion 348 proximate the ring 318 may be configured to cover portions of any flexible barrier having an end portion disposed radially inward of the end portion 348 proximate the ring.

FIG. 11A depicts one embodiment of a medical imaging device 100 and a sterile barrier assembly 301 in a first state. As shown in FIG. 11A, the sterile barrier assembly includes a cap 310 and a barrier housing 320. Like some exemplary embodiments previously discussed, the cap 310 includes a plurality of resilient latches 316 configured to engage a distal portion 104 (e.g., a distal end) of the imaging device 100. In particular, the resilient latches are configured to engage a distal portion of a housing 116 of the imaging device. According to the embodiment of FIGS. 11A-11D, the barrier housing 320 contains a first flexible barrier 340 and a second flexible barrier 342. The first flexible barrier and second flexible barrier are secured to the cap 310. In particular, as shown in FIG. 11A, the first flexible barrier and second flexible barrier are secured to the cap with a ring 318. Of course, other connections are contemplated as described herein, as the present disclosure is not so limited.

In some embodiments, the housing 116 of the imaging device may be coupled to the cap 310 via one or more resilient protrusions. The barrier housing 320 may be held stationary relative to the cap 310 until the one or more resilient protrusions are deflected, for example, by application of force to the cap with the distal portion 104 of the imaging device 100. For example, a user may apply force in a distal direction of the imaging device as shown by the dashed arrow toward the barrier housing 320. The barrier housing 320 may be held by a user, such that a threshold force is applied to the cap 310 to deflect the one or more resilient protrusions and release the cap from the barrier housing 320. In the embodiment of FIG. 11A, the one or more resilient protrusions are configured to deflect to allow insertion of the imaging device 100 through the barrier housing 320. Accordingly, a user may grasp the barrier housing 320 and pull the barrier housing over the imaging device 100 and at least a distal portion of any associated cable extending out from the imaging device to cover the imaging device with one or both of the first flexible barrier 340 and second flexible barrier 342. In the particular embodiment of FIG. 11A, the barrier housing may be pulled over the imaging device to deploy the first flexible barrier 340, as will be shown and described further in reference to FIG. 11B. The second flexible barrier 342 is deployed after the deployment of the first flexible barrier.

FIG. 11B depicts the medical imaging device 100 and sterile barrier assembly of FIG. 11A in a second state. From the state shown in FIG. 11A, a distally directed force was applied to the cap 310 to release the cap from the barrier housing 320. For example, one or more resilient protrusions may deflect from the force applied to the cap, and the cap may be pushed through the barrier housing 320 along with the distal portion 104 of the imaging device 100. As shown in FIG. 11B, the first flexible barrier 340 may have a first end portion 346 and a second end portion 347. The first end portion may be secured to the barrier housing 320 at a flexible barrier connection 349, such that movement of the barrier housing 320 moves the first end portion of the first flexible barrier 340. In some embodiments, the flexible barrier connection 349 may be an ultrasonic weld. Of course, any suitable connection may be employed as described herein, as the present disclosure is not so limited. As shown in FIG. 11B, the second end portion 347 is secured to the cap 310 (e.g., via the ring 318). Accordingly, moving the barrier housing 320 over the imaging device 100 applies tension between the first end portion 346 and the second end portion 347, thereby unrolling and/or unfolding the first flexible barrier 340. The barrier housing may be drawn over the entirety of the imaging device housing 116 as shown in FIG. 11B. The one or more resilient projections present on the barrier housing 320 may deflect to accommodate the insertion of the imaging device through the barrier housing. In some embodiments the barrier housing may be pulled back over the imaging device and an associated cable until the first flexible barrier is fully deployed. In this manner, the barrier housing 320 provides a handle for a user to simply and reliably cover an imaging device and cable with a flexible barrier.

According to the state shown in FIG. 11B, when the first flexible barrier 340 is being deployed (e.g., by passing the imaging device and an associated cable through the barrier housing 320), the second flexible barrier 342 may remain undeployed. That is, the second flexible barrier 342 remains rolled or folded adjacent the cap 310 of the barrier. The deployment of the second flexible barrier may occur after the first flexible barrier is deployed, the process of which is described further in reference to FIG. 11C.

As shown in FIG. 11B. the relative dimensions of the first flexible barrier 340 and the second flexible barrier 342 may be different. As shown in FIG. 11B, the first flexible barrier may have a first maximum transverse dimension T1 (e.g., a maximum diameter in an unstressed state), and the second flexible barrier 342 may have a second maximum transverse dimension T2 (e.g., a maximum diameter in an unstressed state). The first maximum transverse dimension may be greater than the second maximum transverse dimension. In some embodiments, the first maximum transverse dimension may be a factor of 1.25×, 1.5×, 2×, or 3×greater than the second maximum transverse dimension. Of course, any suitable difference in size between the first maximum transverse dimension and the second maximum transverse dimension may be employed, as the present disclosure is not so limited. In the embodiment of FIGS. 11A-11D, the first flexible barrier has a first maximum longitudinal dimension greater than that of a maximum longitudinal dimension of the second flexible barrier. In particular, the first flexible barrier has a length configured to cover the imaging device 100 as well as an associated cable. In the depicted embodiment, the second flexible barrier has a length configured to cover the imaging device only. Of course, in other embodiments any suitable length of a flexible barrier may be employed, as the present disclosure is not so limited.

FIG. 11C depicts the medical imaging device 100 and sterile barrier assembly of FIG. 11A in a third state. As shown in FIG. 11C, the first barrier 340 has been fully deployed and is unrolled and/or unfolded. The state shown in FIG. 11C may correspond to a position where the first flexible barrier is fully extended, and the barrier housing is disposed at a proximal portion of a cable associated with the imaging device. As shown in FIG. 11C, once the first flexible barrier is deployed, the second flexible barrier 342 may be deployed. The second flexible barrier includes an end portion 348 that is also secured to the cap 310 (e.g., via the ring 318). In contrast to the first flexible barrier, the second flexible barrier may have a single end portion exposed, such that the second flexible barrier is unrolled by moving the roll along the housing 116 of the imaging device to unroll the second flexible barrier (e.g., in a proximal direction along at least a portion of the housing of the imaging device). The second flexible barrier 342 is configured to cover the first flexible barrier and the imaging device 100. As the second flexible barrier has a smaller transverse dimension than the first flexible barrier, the second flexible barrier may compress and/or fold the first flexible barrier such that the combined first and second flexible barriers more closely conform to the form of the imaging device housing 116 as compared to the flexible barrier by itself. Accordingly, the second flexible barrier may facilitate manipulation of the imaging device by a user, as less excess material is loosely disposed around the housing 116 of the imaging device.

In some embodiments, deploying the second flexible barrier as shown in FIG. 11C may include stretching (e.g., elastically deforming) the second flexible barrier 342 over the housing 116 of the imaging device. For example, a transverse dimension of the housing 116 of the imaging device may exceed the maximum transverse dimension of the second flexible barrier in an unstressed state. Accordingly, the second flexible barrier may be stressed to stretch the second flexible barrier over the housing 116 of the imaging device. Such an arrangement may apply compression to the first flexible barrier to ensure the combined first and second flexible barriers are compact and fit the form of the imaging device housing 116. In such an embodiment, the second flexible barrier may be formed of an elastic film material, such as latex or polyisoprene. Of course, any suitable material may be employed, as the present disclosure is not so limited. However, embodiments in which the second barrier is not stretched to fit on the imaging device and bands, or other types of connections, may be used to retain the second barrier in a desired shape and placement along a length of the housing of the imaging device as detailed below.

FIG. 11D depicts the medical imaging device 100 and sterile barrier assembly of FIG. 11A in a fourth state. The state of FIG. 11D depicts the barrier in a fully deployed state. As shown in FIG. 11D, the first flexible barrier 340 and the second flexible barrier 342 are fully unrolled and/or unfolded such that they cover the imaging device housing 116. The cap 310 forms a distalmost end of the imaging device 100. The first flexible barrier and second flexible barrier are secured and disposed at a periphery of the cap 310, such that the cap is not covered by the first and second flexible barriers. In some embodiments as shown in FIG. 11D, one or more bands 350, such as elastic bands, may be secured around the first flexible barrier, second flexible barrier, and housing 116 of the imaging device. The elastic bands 350 may apply compression to the first flexible barrier and second flexible barrier to fit the form of the housing 116 of the imaging device. Additionally, the elastic bands 350 may resist longitudinal movement of the first and second flexible barriers so that a user may grasp the second flexible barrier without slip between the first and second barriers, or between the first flexible barrier and the housing 116 of the imaging device. Once in the state of FIG. 11D, the imaging device 100 may be prepared to be used with a patient (e.g., to image a surgical bed).

According to the embodiment of FIG. 11D, once a surgical process is completed, the barrier may be removed by reversing the steps shown and described with reference to FIGS. 11A-11D. For example, the one or more elastic bands 350 may be removed. The second flexible barrier 342 may then be removed from the first flexible barrier 340. A barrier housing 320 may be moved distally to remove the barrier housing and the first flexible barrier from the imaging device housing 116. Finally, a threshold force may be applied to release the cap 310 from the distal portion 104 of the imaging device. Once removed, the barrier assembly may be disposed of. Of course, any suitable process for removal may be employed, as the present disclosure is not so limited.

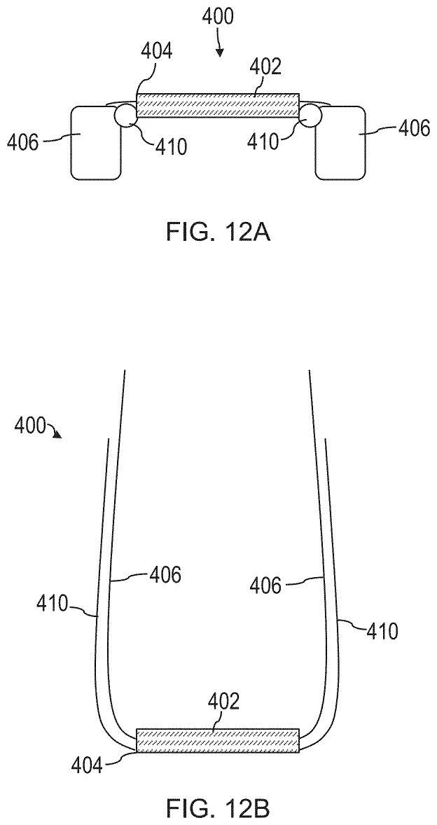

FIG. 12A is a schematic of another embodiment of a sterile barrier assembly 400 in a first state, and FIG. 12B depicts the sterile barrier assembly 400 in a second state. As shown in FIG. 12A, the sterile barrier assembly includes an optically transparent window 402. The window 402 may be configured to be attached to a distal portion of an associated imaging device using a suitable connection (e.g., resilient latches, magnets, etc.). As shown in FIG. 12A, the window is independent and is not formed as part of a cap assembly. Accordingly, in the depicted embodiment, the window functions as a cap. The window includes a periphery 404 which is configured to attach one or more flexible barriers. In the embodiment of FIG. 12A, the barrier assembly includes a first flexible barrier 406 and a second flexible barrier 410. The first flexible barrier and second flexible barrier are both secured to the optically transparent window periphery 404. For example, in some embodiments, the first flexible barrier and second flexible barrier may be ultrasonically welded to the periphery 404 of the window 402. Of course, other suitable connections are contemplated, as the present disclosure is not so limited. Like previously discussed embodiments, the first flexible barrier is configured to be covered by the second flexible barrier, as shown in FIG. 12B. As shown in FIG. 12B, when the first and second flexible barriers are fully extended, the second flexible barrier 410 is disposed radially outward of the first flexible barrier 406. Of course, in other embodiments a single flexible barrier may be employed as a part of a sterile barrier assembly, as the present disclosure is not so limited.

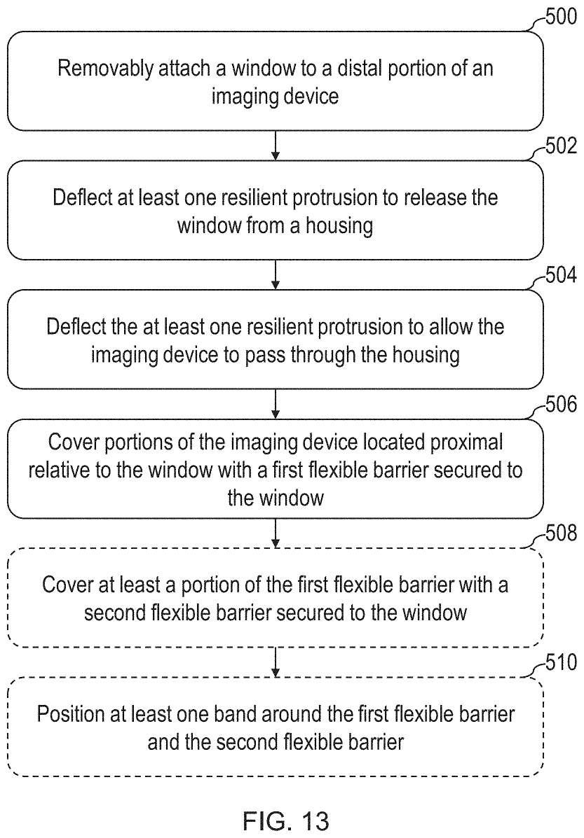

FIG. 13 is a flow chart for one embodiment of a method of installing a sterile barrier on a medical imaging device. In block 500, a window is removably attached to a distal portion of an imaging device. For example, a snap-fit, screw, magnetic, and/or adhesive connection may be employed to releasably attach the window to the distal portion. In some embodiments, the window may be disposed in cap. In block 502, at least one resilient protrusion may be deflected to release the window from a housing. For example, deflecting the at least one resilient protrusion may include disengaging the protrusion from a shelf of the window. In block 504, the at least one resilient protrusion is deflected to allow the imaging device to pass through the housing. For example, the housing may be pulled by a user over the imaging device and an associated cable. In block 506, portions of the imaging device proximal relative to the window are covered with a first flexible barrier. In some embodiments covering the portions of the imaging device proximal relative to the window may include unrolling and/or unfolding the first flexible barrier. In optional block 508, at least a portion of the first flexible barrier is covered with a second flexible barrier secured to the window. In optional block 510, at least one band is positioned around the first flexible barrier and the second flexible barrier.

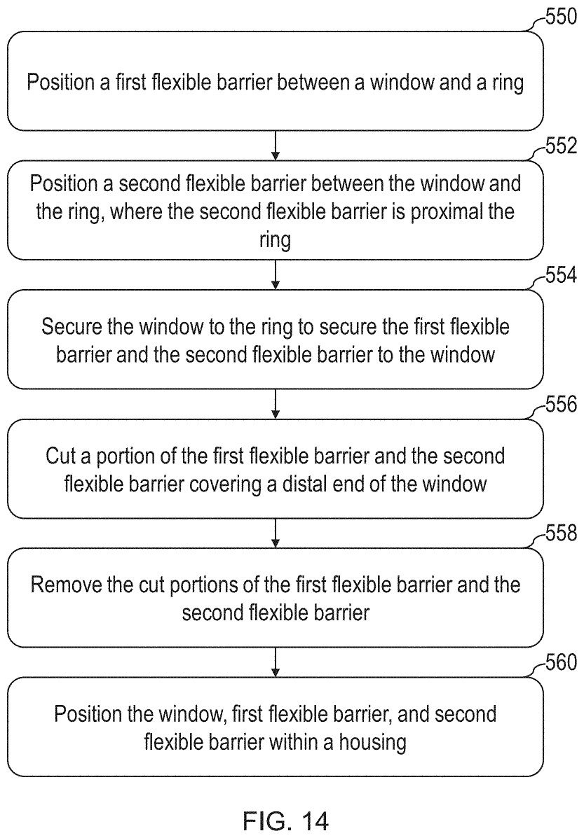

FIG. 14 is a flow chart for one embodiment of a method of assembling a sterile barrier. In block 550, a first flexible barrier is positioned between a window and ring. In block 552, a second flexible barrier is positioned between the window and the ring, where the second flexible barrier is proximal the ring. In block 554, the window is secured to the ring to secure the first flexible barrier and the second flexible barrier to the window. For example, in some embodiments the ring may be press fit onto the window. In block 556, a portion of the first flexible barrier and the second flexible barrier that are disposed over the distal portion of the window are cut. In some embodiments, the first flexible barrier and second flexible barrier may be automatically cut as a result of the securement of the ring to the window. In block 558, the cut portions of the first flexible barrier and the second flexible barrier may be removed. In block 560, the window, first flexible barrier, and second flexible barrier may be positioned within a housing. Of course, while two flexible barriers are employed in the method of FIG. 14, in other embodiments a single flexible barrier, or any other appropriate number of barriers, may be employed, as the present disclosure is not so limited.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A barrier for an imaging device, the barrier comprising:
an optically transparent window configured to be removably attached to a distal portion of the imaging device; and
a flexible barrier secured to the optically transparent window configured to cover portions of the imaging device located proximal relative to the optically transparent window.

2. The barrier of claim 1, wherein the optically transparent window covers the distal portion of the imaging device.

3. The barrier of claim 1, wherein the optically transparent window includes a plurality of resilient latches configured to engage the distal portion of the imaging device.

4. The barrier of claim 1, wherein the optically transparent window is substantially flat.

5. The barrier of claim 1, wherein the flexible barrier is a first flexible barrier, and wherein the barrier further comprises a second flexible barrier secured to the optically transparent window and disposed over at least a portion of the first flexible barrier.

6. The barrier of claim 1, wherein the flexible barrier is secured to a periphery of the optically transparent window.

7. An imaging device comprising the barrier of claim 1, wherein the optically transparent window is removably attached to a distal portion of the imaging device, and wherein a distal side of the optically transparent window is disposed within a depth of field of the imaging device.

8-13. (canceled)

14. A barrier for an imaging device, the barrier comprising:
an optically transparent window including one or more connectors, wherein the optically transparent window is configured to be removably attached to a distal portion of the imaging device; and
a housing containing the optically transparent window, wherein the housing includes at least one resilient protrusion configured to position the optically transparent window in the housing to receive the distal portion of the imaging device.

15. The barrier of claim 14, further comprising a flexible barrier contained in the housing secured to the optically transparent window configured to cover portions of the imaging device located proximal relative to the optically transparent window.

16. The barrier of claim 15, wherein the flexible barrier is secured to the optically transparent window at a first end portion, and wherein the flexible barrier is secured to the housing at a second end portion.

17. The barrier of claim 14, wherein the at least one resilient protrusion is configured to deflect to allow the imaging device to pass through the housing.

18. The barrier of claim 14, wherein the at least one resilient protrusion is a plurality of resilient protrusions.

19. The barrier of claim 18, wherein the plurality of resilient protrusions surrounds the optically transparent window.

20. The barrier of claim 14, wherein the at least one resilient protrusion resists lateral movement of the optically transparent window.

21. The barrier of claim 14, wherein the at least one resilient protrusion is at least one resilient fin.

22. The barrier of claim 21, further comprising a tray configured to support the optically transparent window and resist movement of the optically transparent window in one longitudinal direction.

23. The barrier of claim 22, wherein the tray is engaged with the optically transparent window.

24. A method of installing a barrier on an imaging device, the method comprising:
removably attaching an optically transparent window to a distal portion of the imaging device; and
covering portions of the imaging device located proximal relative to the optically transparent window with a flexible barrier secured to the optically transparent window.

25. The method of claim 24, wherein removably attaching the optically transparent window to the distal portion includes covering the distal portion of the imaging device with the optically transparent window.

26. The method of claim 24, wherein removably attaching the optically transparent window includes engaging the distal portion of the imaging device with a plurality of resilient latches.

27. The method of claim 24, wherein the optically transparent window is substantially flat.

28. The method of claim 24, wherein the flexible barrier is a first flexible barrier, further comprising covering at least a portion of the first flexible barrier with a second flexible barrier secured to the optically transparent window.

29. The method of claim 24, wherein a distal side of the optically transparent window is disposed within a depth of field of the imaging device when removably attached to the distal portion of the imaging device.

30. The method of claim 24, wherein the flexible barrier is secured to a periphery of the optically transparent window.

31-47. (canceled)
